(19) United States
(12) Patent Application Publication
(10) Pub. No.: US 2023/0002357 A1
(43) Pub. Date: Jan. 5, 2023

(54) 7-PYRIMIDINE-2-YL-OXY-INDAZOLE DERIVATIVES AND THEIR USE AS HERBICIDES

(71) Applicant: SYNGENTA CROP PROTECTION AG, Basel (CH)

(72) Inventors: Jeffrey Steven WAILES, Bracknell, Berkshire (GB); James Alan MORRIS, Bracknell, Berkshire (GB); Louisa WHALLEY, Bracknell, Berkshire (GB); Joseph Andrew TATE, Bracknell, Berkshire (GB); Zoe Jane ANDERSON, Bracknell, Berkshire (GB); Sean NG, Bracknell, Berkshire (GB); Mary Bernadette ASPINALL, Bracknell, Berkshire (GB); Catherine Mary HOLDEN, Bracknell, Berkshire (GB)

(73) Assignee: SYNGENTA CROP PROTECTION AG, Basel (CH)

(21) Appl. No.: 17/773,911

(22) PCT Filed: Nov. 12, 2020
(86) PCT No.: PCT/EP2020/081842

§ 371 Date: May 3, 2022

(30) Foreign Application Priority Data
Nov. 14, 2019 (GB) 1916601.6

(51) Int. Cl. C07D 403/12; A01N 43/647; A01N 43/56; A01P 13/00

(52) U.S. Cl. CPC C07D 403/12; A01N 43/647; A01N 43/56; A01P 13/00

(57) ABSTRACT

The present invention relates to compounds of Formula (I), or an agronomically acceptable salt of said compounds wherein X, Y¹, Y², Z¹, Z², R¹, R², R⁵, R⁶ and n are as defined herein. The invention further relates to herbicidal compositions which comprise a compound of Formula (I) and to the use of compounds of Formula (I) for controlling weeds, in particular in crops of useful plants.

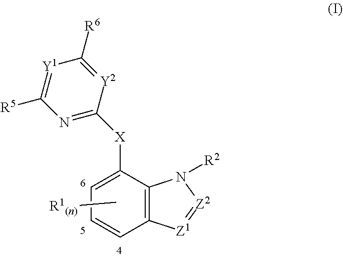

The present invention relates to novel herbicidal compounds, to processes for their preparation, to herbicidal compositions which comprise the novel compounds, and to their use for controlling weeds, in particular in crops of useful plants, or for inhibiting plant growth.

EP0448206A2 discloses various substituted benzimidazole and indazole derivatives and their use as herbicides. EP0367242A2 discloses various aryloxybenzotriazoles and their use as herbicides. The present invention relates to the provision of further herbicidal compounds. Thus, according to the present invention there is provided a compound of Formula (I):

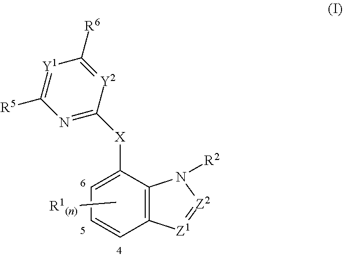

or an agronomically acceptable salt thereof,
wherein
X is selected from the group consisting of CH₂, O or S(O)ₚ;
Y¹ is N or CR³;
Y² is N or CR⁴;
with the proviso that Y¹ and Y² are not both N:
Z¹ is N or CR⁷;
Z² is N or CR⁸;
each R¹ is independently selected from the group consisting of halogen, —CN, nitro, C₁-C₄alkyl, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R² is selected from the group consisting of C₃-C₈ alkyl, C₃-C₈ alkenyl, C₃-C₈ alkynyl, C₃-C₈ haloalkyl, C₃-C₈ haloalkenyl, C₃-C₈ haloalkynyl, C₁-C₄alkoxy-C₁-C₃alkyl-, C₁-C₄haloalkoxy-C₁-C₃alkyl-, C₁-C₄alkoxy-C₁-C₃haloalkyl- and —(CH₂)ₘR⁹
R³ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁴ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁵ is selected from the group consisting of hydrogen, halogen, C₁-C₃alkyl and C₁-C₃haloalkyl;
R⁶ is selected from the group consisting of hydrogen, halogen, C₁-C₃alkyl, C₁-C₃haloalkyl, C₁-C₃alkyl and C₁-C₃haloalkoxy;
R⁷ is selected from the group consisting of hydrogen, halogen, —CN, C₁-C₄alkyl, C₁-C₄haloalkyl- and C₁-C₄alkoxy-;
R⁸ is selected from the group consisting of hydrogen, halogen, —CN, C₁-C₄alkyl, C₁-C₄haloalkyl- and C₁-C₄alkoxy-;
R⁹ is selected from C₃-C₆ cycloalkyl, phenyl and a 5 or 6 membered heteroaryl which comprises from 1 to 4 heteroatoms each independently selected from the group consisting of oxygen, nitrogen and sulphur, and wherein said phenyl or heteroaryl groups are optionally substituted by one, two or three substituents independently selected from the group consisting of halogen, cyano, C₁-C₄alkyl, C₁-C₄haloalkyl, C₁-C₄alkoxy and C₁-C₄haloalkoxy;
m is 1, 2, 3 or 4
n=0, 1 or 2; and
p=0, 1 or 2.

C₁-C₄alkyl- includes, for example, methyl (Me, CH₃), ethyl (Et, C₂H₅), n-propyl (n-Pr), isopropyl (i-Pr), n-butyl (n-Bu), isobutyl (i-Bu), sec-butyl and tert-butyl (t-Bu). C₁-C₂alkyl is methyl (Me, CH₃) or ethyl (Et, C₂H₅).

Halogen (or halo) includes, for example, fluorine, chlorine, bromine or iodine. The same correspondingly applies to halogen in the context of other definitions, such as haloalkyl.

C₁-C₆haloalkyl- includes, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoropropyl and 2,2,2-trichloroethyl, heptafluoro-n-propyl and perfluoro-n-hexyl. C₁-C₄haloalkyl- and C₁-C₂haloalkyl include, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, or 1,1-difluoro-2,2,2-trichloroethyl.

C₁-C₄alkoxy and C₁-C₂alkoxy includes, for example, methoxy and ethoxy.

C₁-C₆haloalkoxy- and C₁-C₄haloalkoxy- include, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy or 2,2,2-trichloroethoxy, preferably difluoromethoxy, 2-chloroethoxy or trifluoromethoxy.

C₂-C₄alkenyl- includes, for example, —CH═CH₂ (vinyl) and —CH₂—CH═CH₂ (allyl).

C₂-C₄alkynyl- refers to a straight or branched hydrocarbon chain radical group consisting solely of carbon and hydrogen atoms, containing at least one triple bond, having from two to four carbon atoms, and which is attached to the rest of the molecule by a single bond. Examples of C₂-C₄alkynyl include, but are not limited to, prop-1-ynyl, propargyl (prop-2-ynyl), and but-1-ynyl.

C₁-C₄alkyl-S— (alkylthio) includes, for example, methylthio, ethylthio, propylthio, isopropylthio, n-butylthio, isobutylthio, sec-butylthio or tert-butylthio, preferably methylthio or ethylthio.

C₁-C₄alkyl-S(O)— (alkylsulfinyl) includes, for example, methylsulfinyl, ethylsulfinyl, propylsulfinyl, isopropylsulfinyl, n-butylsulfinyl, isobutylsulfinyl, sec-butylsulfinyl or tert-butylsulfinyl, preferably methylsulfinyl or ethylsulfinyl.

C₁-C₄alkyl-S(O)₂— (alkylsulfonyl) includes, for example, methylsulfonyl, ethylsulfonyl, propylsulfonyl, isopropylsulfonyl, n-butylsulfonyl, isobutylsulfonyl, sec-butylsulfonyl or tert-butylsulfonyl, preferably methylsulfonyl or ethylsulfonyl.

In one embodiment of the present invention there is provided a compound of Formula (I) wherein X is O or S(O)ₚ. In a further embodiment of the present invention there is provided a compound of Formula (I) wherein X is O. In another embodiment of the present invention there is provided a compound of Formula (I) wherein X is S(O)ₚ (e.g S, S(O) or S(O)₂).

In one embodiment of the present invention there is provided a compound of Formula (I) wherein Y¹ is CR³ and Y² is N (in this embodiment R⁵ and R⁶ are preferably hydrogen) or Y¹ is CR³ and Y² is CR⁴ (in this embodiment R⁵ and R⁶ are preferably hydrogen) or Y¹ is N and Y² is CR⁴ (in this embodiment R⁵ is preferably hydrogen and R⁶ is C₁-C₃alkyl or C₁-C₃alkoxy, more preferably methyl or methoxy).

In another embodiment of the present invention there is provided a compound of Formula (I) wherein Z¹ is CR⁷ and Z² is N or Z¹ is N and Z² is N or Z¹ is N and Z² is CR³. In a more preferred embodiment Z¹ is CR⁷ and Z² is N.

In another embodiment of the present invention there is provided a compound of Formula (I) wherein n=0. In another embodiment of the present invention there is provided a compound of Formula (I) wherein n=1, more preferably wherein R¹ is a substituent in the 6-position, and wherein more preferably 6-halo (especially fluoro or chloro) or 6-CN.

In another embodiment of the present invention there is provided a compound of Formula (I) wherein R² is C₃-C₆alkyl or C₃-C₆haloalkyl, more preferably C₃-C₈fluoroalkyl (e.g CF₃CH₂CH₂CH₂—).

In another embodiment of the present invention there is provided a compound of Formula (I) wherein R³ is halogen, preferably chlorine.

In another embodiment of the present invention there is provided a compound of Formula (I) wherein R⁴ is selected from the group consisting of hydrogen, halogen (e.g fluorine) and —CN. In another embodiment of the present invention there is provided a compound of Formula (I) wherein R⁴ is hydrogen.

In another embodiment of the present invention there is provided a compound of Formula (I) wherein R⁵ is hydrogen.

In another embodiment of the present invention there is provided a compound of Formula (I), wherein R⁶ is selected from the group consisting of hydrogen, C₁-C₄alkyl (preferably methyl) and C₁-C₃alkoxy (preferably methoxy).

In another embodiment of the present invention there is provided a compound of Formula (I), wherein R⁷ is hydrogen or halogen (preferable chlorine).

In another embodiment of the present invention there is provided a compound of Formula (I), wherein R⁸ is hydrogen.

In a particularly preferred embodiment of the present invention there is provided a compound of Formula (I) wherein X is O, Y¹ is CR³ (wherein R³ is preferably chlorine), and Y² is N, R⁵ and R⁶ are hydrogen, Z¹ is CR⁷ (wherein R⁷ is preferably hydrogen or halogen (preferable chlorine) and Z² is N. In this embodiment it is preferred that n is 0 or 1 (wherein R¹=6-F, 6-Cl or 6-CN) and R² is C₃-C₈fluoroalkyl e.g CF₃CH₂CH₂CH₂—.

Compounds of Formula (I) may contain asymmetric centres and may be present as a single enantiomer, pairs of enantiomers in any proportion or, where more than one asymmetric centre are present, contain diastereoisomers in all possible ratios. Typically one of the enantiomers has enhanced biological activity compared to the other possibilities.

The present invention also provides agronomically acceptable salts of compounds of Formula (I). Salts that the compounds of Formula (I) may form with amines, including primary, secondary and tertiary amines (for example ammonia, dimethylamine and triethylamine), alkali metal and alkaline earth metal bases, transition metals or quaternary ammonium bases are preferred.

The compounds of Formula (I) according to the invention can be used as herbicides by themselves, but they are generally formulated into herbicidal compositions using formulation adjuvants, such as carriers, solvents and surface-active agents (SAA). Thus, the present invention further provides a herbicidal composition comprising a herbicidal compound according to any one of the previous claims and an agriculturally acceptable formulation adjuvant. The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The herbicidal compositions generally comprise from 0.1 to 99% by weight, especially from 0.1 to 95% by weight, compounds of Formula I and from 1 to 99.9% by weight of a formulation adjuvant which preferably includes from 0 to 25% by weight of a surface-active substance.

The compositions can be chosen from a number of formulation types. These include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a soluble powder (SP), a wettable powder (WP) and a soluble granule (SG). The formulation type chosen in any instance will depend upon the particular purpose envisaged and the physical, chemical and biological properties of the compound of Formula (I).

Soluble powders (SP) may be prepared by mixing a compound of Formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of Formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of Formula (I) and one or more powdered solid diluents or carriers, or from pre-formed blank granules by absorbing a compound of Formula (I) (or a solution thereof, in a suitable agent) in a porous granular material (such as pumice, attapulgite clays, fuller's earth, kieselguhr, diatomaceous earths or ground corn cobs) or by adsorbing a compound of Formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of Formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of Formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of Formula (I) either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SAAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SAAs, to produce spontaneously a thermodynamically stable isotropic liquid formulation. A compound of Formula (I) is present initially in either the water or the solvent/SAA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of Formula (I). SCs may be prepared by ball or bead milling the solid compound of Formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of Formula (I) may be dry milled and added to water, containing agents hereinbefore described, to produce the desired end product.

Aerosol formulations comprise a compound of Formula (I) and a suitable propellant (for example n-butane). A compound of Formula (I) may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of Formula (I) and, optionally, a carrier or diluent therefor. The polymeric shell may be produced by either an interfacial polycondensation reaction or by a coacervation procedure. The compositions may provide for controlled release of the compound of Formula (I) and they may be used for seed treatment. A compound of Formula (I) may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of Formula (I). Such additives include surface active agents (SAAs), spray additives based on oils, for example certain mineral oils or natural plant oils (such as soy bean and rape seed oil), modified plant oils such as methylated rape seed oil (MRSO), and blends of these with other bio-enhancing adjuvants (ingredients which may aid or modify the action of a compound of Formula (I).

Wetting agents, dispersing agents and emulsifying agents may be SAAs of the cationic, anionic, amphoteric or non-ionic type.

Suitable SAAs of the cationic type include quaternary ammonium compounds (for example cetyltrimethyl ammonium bromide), imidazolines and amine salts.

Suitable anionic SAAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates, lignosulphonates and phosphates/sulphates of tristyrylphenols.

Suitable SAAs of the amphoteric type include betaines, propionates and glycinates.

Suitable SAAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); lecithins and sorbitans and esters thereof, alkyl polyglycosides and tristyrylphenols.

Suitable suspending agents include hydrophilic colloids (such as polysaccharides, polyvinylpyrrolidone or sodium carboxymethylcellulose) and swelling clays (such as bentonite or attapulgite).

The herbicidal compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, bensulfuron (including bensulfuron-methyl), bentazone, bicyclopyrone, bilanafos, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, diquat dibromide, diuron, ethalfluralin, ethofumesate, fenoxaprop (including fenoxaprop-P-ethyl), fenoxasulfone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, flupyrsulfuron (including flupyrsulfuron-methyl-sodium), fluroxypyr (including fluroxypyr-meptyl), fomesafen, foramsulfuron, glufosinate (including the ammonium salt thereof), glyphosate (including the diammonium, isopropylammonium and potassium salts thereof), halauxifen (including halauxifen-methyl), haloxyfop (including haloxyfop-methyl), hexazinone, hydantocidin, imazamox, imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, ethyl 2-[[3-[2-chloro-4-fluoro-5-[3-methyl-2,6-dioxo-4-(trifluoromethyl)pyrimidin-1-yl]phenoxy]-2-pyridyl]oxy]acetate, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 3-[2-(3,4-dimethoxyphenyl)-6-methyl-3-oxo-pyridazine-4-carbonyl]bicyclo[3.2.1]octane-2,4-dione, 2-[2-(3,4-dimethoxyphenyl)-6-methyl-3-oxo-pyridazine-4-carbonyl]-5-methyl-cyclohexane-1,3-dione, 2-[2-(3,4-dimethoxyphenyl)-6-methyl-3-oxo-pyridazine-4-carbonyl]cyclohexane-1,3-dione, 2-[2-(3,4-dimethoxyphenyl)-6-methyl-3-oxo-pyridazine-4-carbonyl]-5,5-dimethyl-cyclohexane-1,3-dione, 6-[2-(3,4-dimethoxyphenyl)-6-methyl-3-oxo-pyridazine-4-carbonyl]-2,2,4,4-tetramethyl-cyclohexane-1,3,5-trione, 2-[2-(3,4-dimethoxyphenyl)-6-methyl-3-oxo-pyridazine-4-carbonyl]-5-ethyl-cyclohexane-1,3-dione, 2-[2-(3,4-dimethoxyphenyl)-6-methyl-3-oxo-pyridazine-4-carbonyl]-4,4,6,6-tetramethyl-cyclohexane-1,3-dione, 2-[6-cyclopropyl-2-(3,4-dimethoxyphenyl)-3-oxo-pyridazine-4-carbonyl]-5-methyl-cyclohexane-1,3-dione, 3-[6-cyclopropyl-2-(3,4-dimethoxyphenyl)-3-oxo-pyridazine-4-carbonyl]bicyclo[3.2.1]octane-2,4-dione, 2-[6-cyclopropyl-2-(3,4-dimethoxyphenyl)-3-oxo-pyridazine-4-carbonyl]-5,5-dimethyl-cyclohexane-1,3-dione, 6-[6-cyclopropyl-2-(3,4-dimethoxyphenyl)-3-oxo-pyridazine-4-carbonyl]-2,2,4,4-tetramethyl-cyclohexane-1,3,5-trione, 2-[6-cyclopropyl-2-(3,4-dimethoxyphenyl)-3-oxo-pyridazine-4-carbonyl]cyclohexane-1,3-dione, 4-[2-(3,4-dimethoxyphenyl)-6-methyl-3-oxo-pyridazine-4-carbonyl]-2,2,6,6-tetramethyl-tetrahydropyran-3,5-dione, 4-[6-cyclopropyl-2-(3,4-dimethoxyphenyl)-3-oxo-pyridazine-4-carbonyl]-2,2,6,6-tetramethyl-tetrahydropyran-3,5-dione and 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate).

The mixing partners of the compound of Formula (I) may also be in the form of esters or salts, as mentioned e.g. in The Pesticide Manual, Sixteenth Edition, British Crop Protection Council, 2012.

The compound of Formula (I) can also be used in mixtures with other agrochemicals such as fungicides, nematicides or insecticides, examples of which are given in The Pesticide Manual.

The mixing ratio of the compound of Formula (I) to the mixing partner is preferably from 1:100 to 1000:1.

The mixtures can advantageously be used in the above-mentioned formulations (in which case “active ingredient” relates to the respective mixture of compound of Formula (I) with the mixing partner).

The compounds or mixtures of the present invention can also be used in combination with one or more herbicide safeners. Examples of such safeners include benoxacor, cloquintocet (including cloquintocet-mexyl), cyprosulfamide, dichlormid, fenchlorazole (including fenchlorazole-ethyl), fenclorim, fluxofenim, furilazole, isoxadifen (including isoxadifen-ethyl), mefenpyr (including mefenpyr-diethyl), metcamifen and oxabetrinil.

Particularly preferred are mixtures of a compound of Formula (I) with cyprosulfamide, isoxadifen-ethyl, cloaquintocet-mexyl and/or metcamifen.

The safeners of the compound of Formula (I) may also be in the form of esters or salts, as mentioned e.g. in The Pesticide Manual, 16^{th} Edition (BCPC), 2012. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048.

Preferably the mixing ratio of compound of Formula (I) to safener is from 100:1 to 1:10, especially from 20:1 to 1:1.

The present invention still further provides a method of controlling weeds at a locus said method comprising application to the locus of a weed controlling amount of a composition comprising a compound of Formula (I). Moreover, the present invention may further provide a method of selectively controlling weeds at a locus comprising crop plants and weeds, wherein the method comprises application to the locus of a weed controlling amount of a composition according to the present invention. ‘Controlling’ means killing, reducing or retarding growth or preventing or reducing germination. It is noted that the compounds of the present invention show a much improved selectivity compared to know, structurally similar compounds.

Generally the plants to be controlled are unwanted plants (weeds). ‘Locus’ means the area in which the plants are growing or will grow. The application may be applied to the locus pre-emergence and/or postemergence of the crop plant. Some crop plants may be inherently tolerant to herbicidal effects of compounds of Formula (I). Preferred crop plants include maize, wheat, barley and rice.

The rates of application of compounds of Formula I may vary within wide limits and depend on the nature of the soil, the method of application (pre- or post-emergence; seed dressing; application to the seed furrow; no tillage application etc.), the crop plant, the weed(s) to be controlled, the prevailing climatic conditions, and other factors governed by the method of application, the time of application and the target crop. The compounds of Formula I according to the invention are generally applied at a rate of from 10 to 2500 g/ha, especially from 25 to 1000 g/ha, more especially from 25 to 250 g/ha.

The application is generally made by spraying the composition, typically by tractor mounted sprayer for large areas, but other methods such as dusting (for powders), drip or drench can also be used.

Crop plants are to be understood as also including those crop plants which have been rendered tolerant to other herbicides or classes of herbicides (e.g. ALS-, GS-, EPSPS-, PPO-, HPPD-, -PDS and ACCase-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crop plants are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crop plants are also to be understood to include those which are obtained by conventional methods of breeding or genetic engineering and contain so-called output traits (e.g. improved storage stability, higher nutritional value and improved flavour).

The compositions can be used to control unwanted plants (collectively, ‘weeds’). The weeds to be controlled may be both monocotyledonous species, for example Agrostis, Alopecurus, Avena, Brachiaria, Bromus, Cenchrus, Cyperus, Digitaria, Echinochloa, Eleusine, Lolium, Monochoria, Rottboellia, Sagittaria, Scirpus, Setaria and Sorghum, and dicotyledonous species, for example Abutilon, Amaranthus, Ambrosia, Chenopodium, Chrysanthemum, Conyza, Galium, Ipomoea, Nasturtium, Sida, Sinapis, Solanum, Stellaria, Veronica, Viola and Xanthium.

In a further aspect of the present invention there is provided the use of a compound of Formula (I) as defined herein as a herbicide.

Processes for preparation of compounds, e.g. a compound of formula (I) (which optionally can be an agrochemically acceptable salt thereof), are now described, and form further aspects of the present invention.

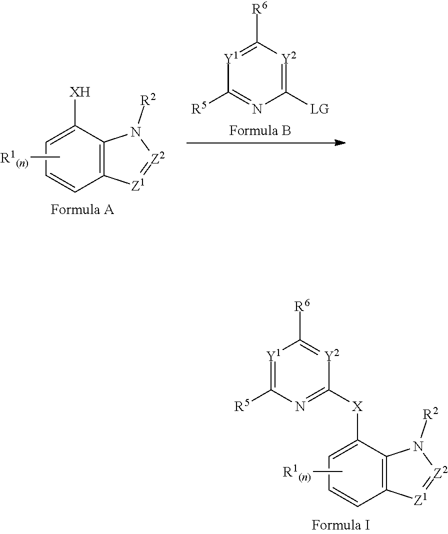

A compound of Formula I may be prepared from a compound of Formula A by reaction with a compound of Formula B (where LG represents a suitable leaving group such as halogen or SO₂R) optionally in the presence of a suitable base and in a suitable solvent. Suitable bases may include K₂CO₃ or Cs₂CO₃. Suitable solvents may include DMF. Compounds of Formula B are commercially available or may be prepared by methods known in the literature.

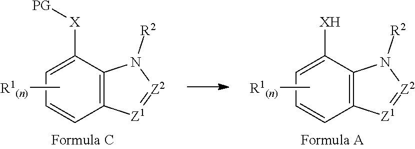

A compound of Formula A may be prepared from a compound of Formula C (where PG represents a suitable protecting group, such as Me) via a deprotection reaction using a suitable reagent in a suitable solvent. When PG=Me, suitable reagents for carrying out this deprotection may include BBr₃, or dodecanethiol/LiO^{t}Bu. Suitable solvents may include DCM or DMF.

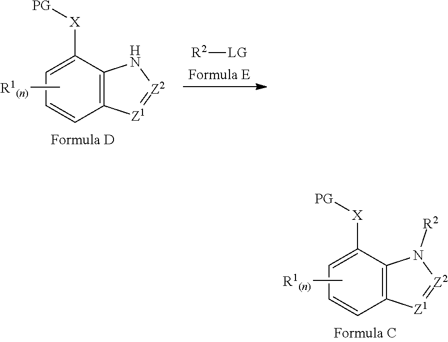

A compound of Formula C may be prepared from a compound of Formula D via reaction with a compound of Formula E (where LG represents a suitable leaving group such as Cl, Br or I or a sulfonate derivative such as Oms, OTs or OTf), optionally in the presence of a suitable base and in a suitable solvent. Suitable bases may include Cs₂CO₃, K₂CO₃ or NaH. Suitable solvents may include DMF, THF or CH₃CN. Compounds of Formula D and of Formula E are commercially available or may be prepared by methods known in the literature.

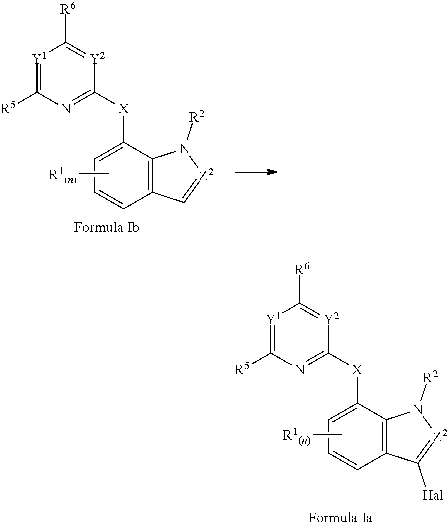

Alternatively, a compound of Formula Ia (a compound of Formula I where Z¹=C-Hal (where Hal represents a halogen) may be prepared from a compound of Formula Ib (a compound of Formula I where Z¹═C—H) via a halogenation reaction with a suitable reagent in a suitable solvent. Suitable halogenation reagents may include N-chloro-succinimide. Suitable solvents may include CH₃CN.

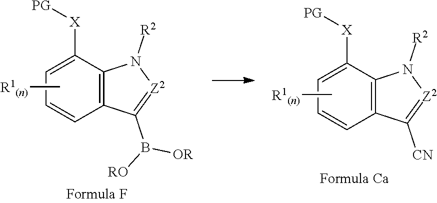

In an alternative process, a compound of Formula Ca (a compound of Formula C where Z¹═C—CN) may be prepared from a compound of Formula F via a cyanation reaction using a suitable cyanide source in the presence of a suitable catalyst and a suitable base and in a suitable solvent. Suitable cyanide sources may include Zn(CN)₂. Suitable catalysts may include Cu(NO₃)₂. Suitable bases may include CsF. Suitable solvents may include MeOH/water mixtures.

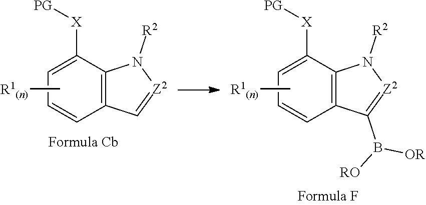

A compound of Formula F may be prepared from a compound of Formula Cb (a compound of Formula C where Z¹═C—H) via a borylation reaction in the presence of a suitable boron reagent and a suitable catalyst and a suitable ligand and in a suitable solvent. Suitable boron reagents may include bis(pinacolato)diboron. Suitable catalysts may include (1,5-cyclooctadiene)(methoxy)iridium(I) dimer. Suitable ligands may include 4,4′-di-tert-butyl-2,2′-bipyridine. Suitable solvents may include tert-butyl methyl ether.

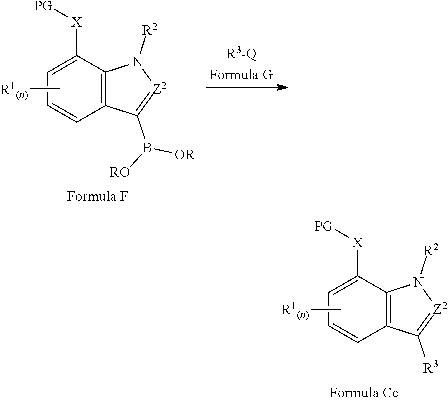

In a yet further alternative process, a compound of Formula Cc (a compound of Formula C where Z¹═C—R³ (where R³═C₁₋₄ alkyl) may be prepared by reaction of a compound of Formula F with a compound of Formula G (where Q represents a suitable cross-coupling functional group such as halogen or pseudohalogen) in the presence of a suitable catalyst and a suitable ligand, optionally in the presence of a suitable base and in a suitable solvent. Suitable catalysts may include Pd(OAc)₂. Suitable ligands may include di-tertbutyl(methyl)phosphonium tetrafluoroborate. Suitable bases may include K₂CO₃. Suitable solvents may include 2-methyl-2-butanol.

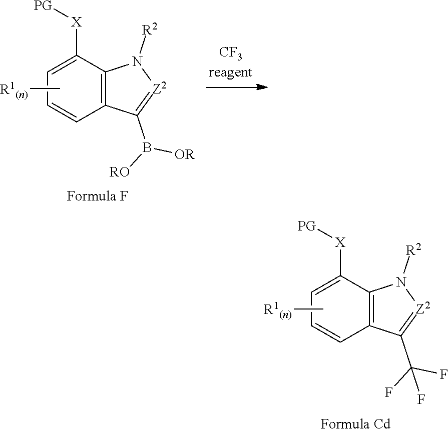

In another yet further alternative process, a compound of Formula Cd (a compound of Formula C where Z¹═C—CF₃) may be prepared by reaction of a compound of Formula F with a suitable CF₃ reagent, optionally in the presence of a suitable base and in a suitable solvent. Suitable CF₃ reagents may include (1,10-phenanthroline)(trifluoromethyl)copper(I). Suitable bases may include KF. Suitable solvents may include DMF.

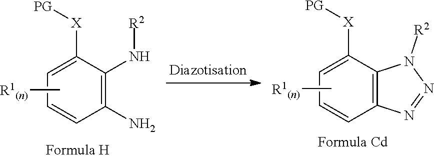

In a yet further alternative process, a compound of Formula Cd (a compound of Formula C where Z¹ and Z²═N) may be prepared via a diazotisation/cyclisation reaction using a suitable diazotising reagent and in the presence of a suitable acid. Suitable diazotising reagents may include NaNO₂. Suitable acids may include hydrochloric acid.

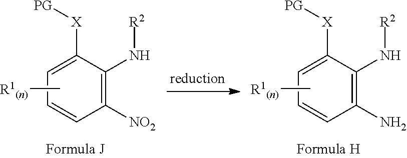

A compound of Formula H may be prepared from a compound of Formula J via a reduction reaction in a suitable solvent. Suitable reduction conditions may include H₂/Pd—C. Suitable solvents may include EtOH or MeOH.

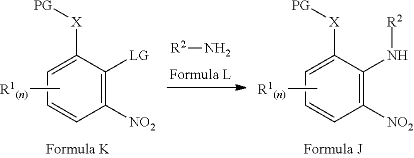

A compound of Formula J may be prepared from a compound of Formula K (where LG represents a suitable leaving group such as Cl or F) via an S_{N}^{AR} reaction with a compound of Formula L optionally in the presence of a suitable base and in a suitable solvent. Suitable bases may include N,N-diisopropylethylamine. Suitable solvents may include NMP. Compounds of Formula K and of Formula L are commercially available or may be prepared by known methods.

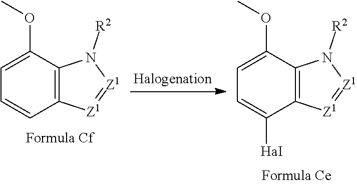

In a yet further alternative process, a compound of Formula Ce (a compound of Formula C where X═O, PG=Me and R¹=4-halogen) may be prepared from a compound of Formula Cf (a compound of Formula C where X═O, PG=Me and n=0) via reaction with a suitable halogenating reagent in a suitable solvent. Suitable halogenating reagents may include sulfuryl chloride. Suitable solvents may include DCM.

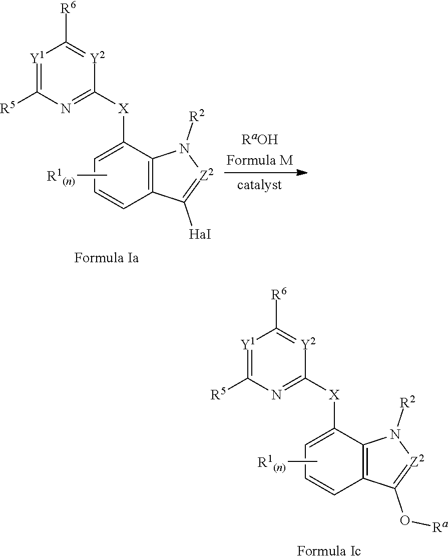

In a yet further alternative process, a compound of Formula Ic (a compound of Formula I where Z¹═C—OR^{a}, where R^{a}═C₁₋₄ alkyl) may be prepared from a compound of Formula Ia (a compound of Formula I where Z¹=halogen, preferably Br) by reaction with a compound of Formula M in the presence of a suitable catalyst and a suitable base and in a suitable solvent. Suitable catalysts may include Rockphos Pd G3. Suitable bases may include caesium carbonate. Suitable solvents may include 1,4-dioxane. Compounds of Formula M are commercially available or may be prepared by known methods.

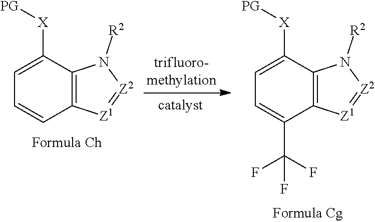

In a still further alternative process, a compound of Formula Cg (a compound of Formula C where R¹=4-CF₃) may be prepared from a compound of Formula Ch (a compound of Formula C where n=0) via a radical trifluoromethylation using a suitable CF₃ precursor in the presence of a suitable catalyst and a suitable oxidant, in a suitable solvent and irradiated with suitable light. Suitable trifluoromethyl precursors may include (2,2,2-trifluoroacetyl) 2,2,2-trifluoroacetate. Suitable catalysts may include tris(2,2′-bipyridyl)dichlororuthenium(II) hexahydrate. Suitable oxidants may include pyridine-N-oxide. Suitable solvents may include acetonitrile.

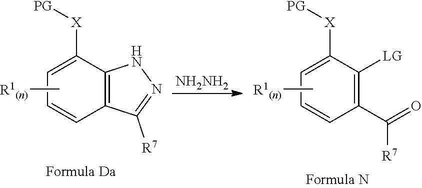

In a still yet further alternative process, a compound of Formula Da (a compound of Formula D where Z¹═C—R⁷ and Z²═N) may be prepared from a compound of Formula N (where LG represents a suitable leaving group such as F or Cl) via a flow cyclisation with hydrazine in a suitable solvent. Suitable solvents may include 1,4-dioxane.

The following non-limiting examples provide specific synthesis methods for representative compounds of the present invention, as referred to in the Table below.

### EXAMPLE 1: SYNTHESIS OF 7-(5-CHLOROPYRIMIDIN-2-YL)OXY-1-(4,4,4-TRIFLUOROBUTYL)INDAZOLE (1.001)

### Step 1: Synthesis of 7-methoxy-1-(4,4,4-trifluorobutyl)indazole

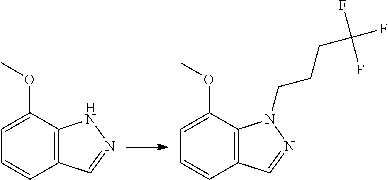 [SMILES: COc1cccc2cn[nH]c12 ; COc1cccc2cnn(CCCC(F)(F)F)c12]

A solution of 7-methoxy-1H-indazole (100 mg, 0.67 mmol), Cs₂CO₃ (440 mg, 2.3 mmol) and 4-bromo-1,1,1-trifluoro-butane (200 mg, 1.0 mmol) in DMF (5 mL) was stirred at RT 30 mins and then heated at 80° C. for 30 minutes under microwave irradiation. The reaction mixture was diluted with water and extracted with Et₂O. The combined organic extracts were washed with water and brine, dried over MgSO₄ and evaporated to dryness under reduced pressure. The crude product was purified by flash chromatography on silica gel using a gradient of EtOAc in cyclohexane to give the desired product (150 mg, 86%) as a colourless oil.

¹H NMR (500 MHz, CDCl₃) δ 7.92 (s, 1H) 7.27 (t, 1H) 7.04 (t, 1H) 6.72 (d, 1H) 4.72 (t, 2H) 3.98 (s, 3H) 2.21-2.03 (m, 4H).

### Step 2: Synthesis of 1-(4,4,4-trifluorobutyl)indazol-7-ol

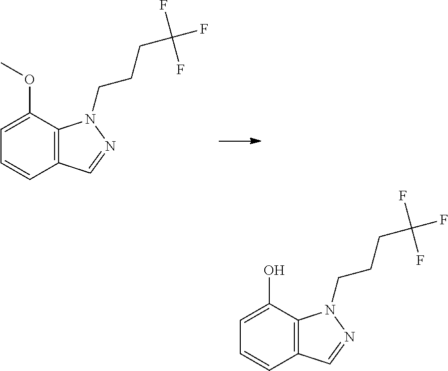 [SMILES: COc1cccc2cnn(CCCC(F)(F)F)c12 ; Oc1cccc2cnn(CCCC(F)(F)F)c12]

To a solution of 7-methoxy-1-(4,4,4-trifluorobutyl)indazole (1.9 g, 7.4 mmol) in DMF (50 mL) at RT under an atmosphere of N2 was added dodecane-1-thiol (3.3 mL, 14 mmol) followed by a solution of lithium t-butoxide (1M in THF) (14 mL, 14 mmol). The reaction was heated at 100° C. for 4 hours and then allowed to cool to RT. The reaction mixture was diluted with 2M HCl and extracted into Et₂O. The combined organic extracts were washed with water and brine, dried over MgSO₄ and evaporated to dryness under reduced pressure. The crude product was purified by flash chromatography on silica gel using a gradient of EtOAc in cyclohexane to give the desired product (1.70 g, 95%) as a white waxy solid.

¹H NMR (400 MHz, CDCl₃) δ 7.99 (s, 1H) 7.29 (t, 1H) 6.96 (t, 1H) 6.70 (d, 1H) 6.68 (s, 1H) 4.78 (t, 2H) 2.26-2.04 (m, 4H).

### Step 3: Synthesis of 7-(5-chloropyrimidin-2-yl)oxy-1-(4,4,4-trifluorobutyl)indazole (1.001)

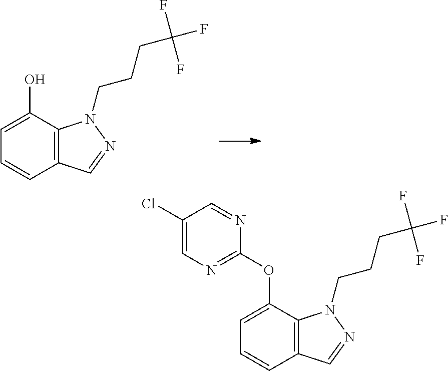 [SMILES: Oc1cccc2cnn(CCCC(F)(F)F)c12 ; FC(F)(F)CCCn1ncc2cccc(Oc3ncc(Cl)cn3)c21]

To a solution of 1-(4,4,4-trifluorobutyl)indazol-7-ol (200 mg, 0.82 mmol) in DMF (10 mL) at RT was added 2,5-dichloropyrimidine (140 mg, 0.91 mmol) and K₂CO₃ (300 mg, 2.1 mmol). The reaction was heated to 80° C. for 1 hour and then allowed to cool to RT. The reaction mixture was diluted with 2M HCl and extracted with Et₂O. The combined organic extracts were washed with water and brine, dried over MgSO₄ and evaporated to dryness under reduced pressure. The crude product was purified by flash chromatography on silica gel using a gradient of EtOAc in cyclohexane as eluent to give the desired product (265 mg, 91%) as a pale yellow gum.

¹H NMR (400 MHz, CDCl₃) δ 8.53 (s, 2H), 8.05 (s, 1H), 7.68-7.62 (m, 1H), 7.20-7.15 (m, 2H), 4.48 (br t, 2H), 2.10-198 (m, 2H).

### EXAMPLE 2: SYNTHESIS OF 3-CHLORO-7-(5-CHLOROPYRIMIDIN-2-yl)oxy-1-(4,4,4-Trifluorobutyl)indazole (1.005)

### Step 1: Synthesis of 3-chloro-7-(5-chloropyrimidin-2-yl)oxy-1-(4,4,4-trifluorobutyl)indazole (1.005)

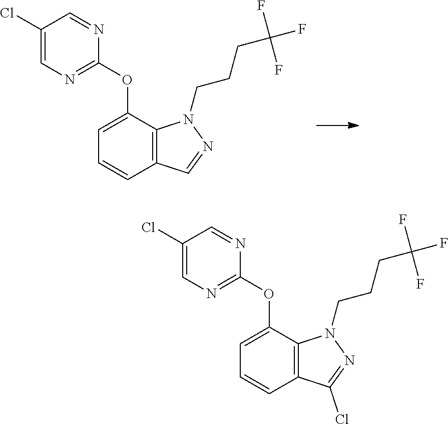 [SMILES: FC(F)(F)CCCn1ncc2cccc(Oc3ncc(Cl)cn3)c21 ; FC(F)(F)CCCn1nc(Cl)c2cccc(Oc3ncc(Cl)cn3)c21]

A solution of 7-(5-chloropyrimidin-2-yl)oxy-1-(4,4,4-trifluorobutyl)indazole (0.67 g, 1.88 mmol) and N-chlorosuccinimide (0.276 g, 2.07 mmol) in CH₃CN (17 mL) was heated under microwave irradiation at 80° C. for 2 hours. Further N-chlorosuccinimide (200 mg) was added and the reaction was heated at 80° C. under microwave irradiation for a further 2 hours. The reaction mixture was cooled to RT and then evaporated to dryness under reduced pressure. The crude product was purified by flash chromatography on silica gel using a gradient of 0-80% EtOAc in cyclohexane as eluent to give the desired product (181 mg, 25%) as a colourless gum.

¹H NMR (400 MHz, CDCl₃) δ 8.53 (s, 2H), 7.62-7.57 (m, 1H), 7.25-7.20 (m, 2H), 4.42 (br t, 2H), 2.10-1.98 (m, 4H).

### EXAMPLE 3: SYNTHESIS OF 7-(5-CHLOROPYRIMIDIN-2-YL)OXY-1-(4,4,4-TRIFLUOROBUTYL)INDAZOLE-3-CARBONITRILE (1.007)

### Step 1: Synthesis of 7-methoxy-3-(4,4,5,5-tetramethyl-1,3,2-dioxaborolan-2-yl)-1-(4,4,4-trifluorobutyl)indazole

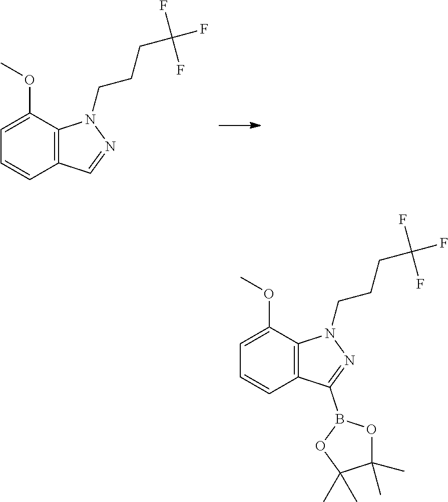 [SMILES: COc1cccc2cnn(CCCC(F)(F)F)c12 ; COc1cccc2c(B3OC(C)(C)C(C)(C)O3)nn(CCCC(F)(F)F)c12]

To a solution of bis(pinacolato)diboron (0.596 g, 2.32 mmol), 4,4′-di-tert-butyl-2,2′-bipyridine (0.032 g, 0.116 mmol) and (1,5-cyclooctadiene)(methoxy)iridium(1) dimer (0.039 g, 0.0581 mmol) in tert-butyl methyl ether (5.8 mL) was added 7-methoxy-1-(4,4,4-trifluorobutyl)indazole (0.50 g, 1.94 mmol). The reaction was heated at 80° C. under microwave irradiation for 1 hour. The reaction mixture was evaporated to dryness under reduced pressure and the crude product purified by flash chromatography on silica gel using a gradient 0-10% EtOAc in cyclohexane as eluent to give the desired product (0.45 g, 61%) as a colourless gum.

¹H NMR (400 MHz, CDCl₃) δ 7.65 (d, 1H), 7.09 (t, 1H), 6.72 (d, 1H), 4.79 (t, 2H), 3.97 (s, 3H), 2.23-2.05 (m, 4H), 1.41 (s, 12H).

### Step 2: Synthesis of 7-methoxy-1-(4,4,4-trifluorobutyl)indazole-3-carbonitrile

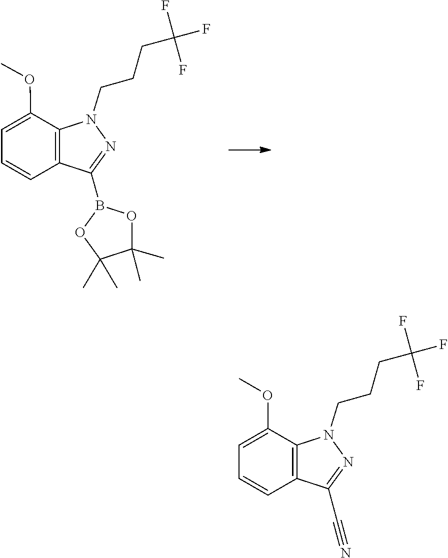 [SMILES: COc1cccc2c(B3OC(C)(C)C(C)(C)O3)nn(CCCC(F)(F)F)c12 ; COc1cccc2c(C#N)nn(CCCC(F)(F)F)c12]

A solution of 7-methoxy-3-(4,4,5,5-tetramethyl-1,3,2-dioxaborolan-2-yl)-1-(4,4,4-trifluorobutyl)indazole (0.285 g, 0.742 mmol), Cu(NO₃)₂.3H₂O (0.272 g, 1.11 mmol), Zn(CN)₂ (0.269 g, 2.23 mmol) and CsF (0.114 g, 0.742 mmol) in MeOH (3.7 mL) and water (1.5 mL) were heated at 100° C. under microwave irradiation for 1 hour. The reaction mixture was diluted with sat. ammonium chloride and extracted with EtOAc. The combined organics were washed with water, brine, dried over MgSO₄ and evaporated to dryness under reduced pressure. The crude product was purified by flash chromatography on silica gel using a gradient of 0-20% EtOAc/cyclohexane as eluent to give the desired product (100 mg, 48%) as a colourless solid.

¹H NMR (400 MHz, CDCl₃) δ 7.38 (d, 1H), 7.25 (t, 1H), 6.82 (d, 1H), 4.78 (t, 2H), 4.01 (s, 3H), 2.27-2.07 (m, 4H).

### Step 3: Synthesis of 7-hydroxy-1-(4,4,4-trifluorobutyl)indazole-3-carbonitrile

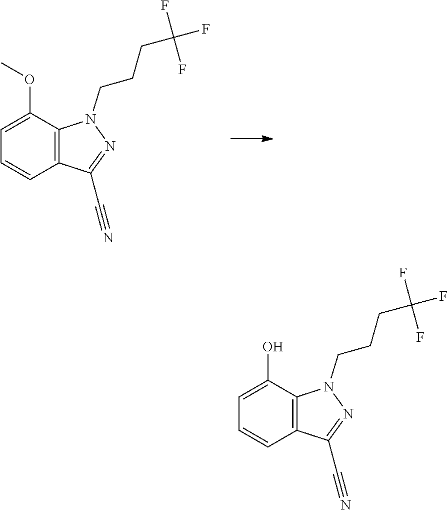 [SMILES: COc1cccc2c(C#N)nn(CCCC(F)(F)F)c12 ; N#Cc1nn(CCCC(F)(F)F)c2c(O)cccc12]

To a solution of 7-methoxy-1-(4,4,4-trifluorobutyl)indazole-3-carbonitrile (0.100 g, 0.353 mmol) and 1-dodecanethiol (0.146 g, 0.706 mmol) in DMF (1 mL) was added lithium t-butoxide (1M in THF) (0.71 mL, 0.71 mmol) The reaction was heated at 100° C. for 2 hours, cooled to RT and evaporated to dryness under reduced pressure. The crude product was purified by flash chromatography on silica gel using a gradient of 0-30% EtOAc/cyclohexane as eluent to give the impure desired product (105 mg) as a pale yellow oil which was used without further purification.

¹H NMR (400 MHz, CDCl₃) δ 7.57 (s, 1H), 7.34 (d, 1H), 7.14 (t, 1H), 6.81 (d, 1H), 4.81 (t, 2H), 2.31-2.08 (m, 4H).

### Step 4: Synthesis of 7-(5-chloropyrimidin-2-yl)oxy-1-(4,4,4-trifluorobutyl)indazole-3-carbonitrile (1.007)

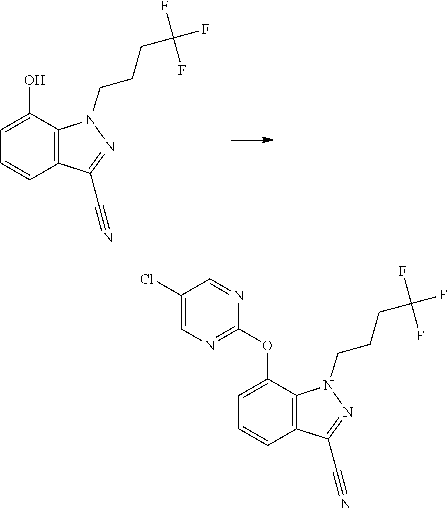 [SMILES: N#Cc1nn(CCCC(F)(F)F)c2c(O)cccc12 ; N#Cc1nn(CCCC(F)(F)F)c2c(Oc3ncc(Cl)cn3)cccc12]

To a mixture of 7-hydroxy-1-(4,4,4-trifluorobutyl)indazole-3-carbonitrile (105 mg, 0.390 mmol) and K₂CO₃ (108 mg 0.78 mmol) in DMF (1 mL) was added 2,5-dichloropyrimidine (71 mg, 0.468 mmol). The reaction was heated at 70° C. for 20 hours, allowed to cool to RT and evaporated to dryness under reduced pressure. The crude product was purified by flash chromatography on silica gel using a gradient of 0-15% EtOAc/cyclohexane as eluent (125 mg, 84%) as a colourless solid.

¹H NMR (400 MHz, CDCl₃) δ 8.54 (s, 2H), 7.76 (dd, 1H), 7.37 (t, 1H), 7.31 (dd, 1H), 4.58 (t, 2H), 2.17-2.05 (m, 4H).

### EXAMPLE 4: SYNTHESIS OF 7-(5-CHLOROPYRIMIDIN-2-YL)OXY-3-METHYL-1-(4,4,4-TRIFLUOROBUTYL)INDAZOLE (1.021)

### Step 1: Synthesis of 7-methoxy-3-methyl-1-(4,4,4-trifluorobutyl)indazole

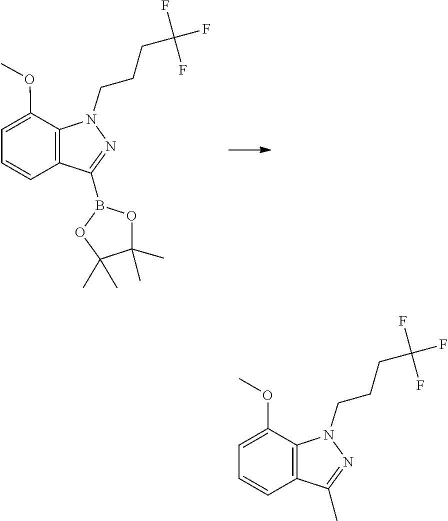 [SMILES: COc1cccc2c(B3OC(C)(C)C(C)(C)O3)nn(CCCC(F)(F)F)c12 ; COc1cccc2c(C)nn(CCCC(F)(F)F)c12]

A mixture of Pd(OAc)₂ (0.011 g, 0.0651 mmol), di-tertbutyl(methyl)phosphonium tetrafluoroborate (0.033 g, 0.130 mmol), K₂CO₃ (0.216 g, 1.56 mmol) and 7-methoxy-3-(4,4,5,5-tetramethyl-1,3,2-dioxaborolan-2-yl)-1-(4,4,4-trifluorobutyl)indazole (0.500 g, 1.30 mmol) in 2-methyl-2-butanol (13 mL) was degassed and purged with nitrogen three times. Iodomethane (0.371 g, 2.60 mmol) was added and the reaction heated at 65° C. for 24 hours. The reaction was cooled to RT and evaporated to dryness under reduced pressure. The crude product was purified by flash chromatography on silica gel using a gradient of 0-20% EtOAc/cyclohexane as eluent to give the desired product as an inseparable mixture with 7-methoxy-1-(4,4,4-trifluorobutyl)indazole which was used in the next step without further purification.

### Step 2: Synthesis of 3-methyl-1-(4,4,4-trifluorobutyl)indazol-7-ol

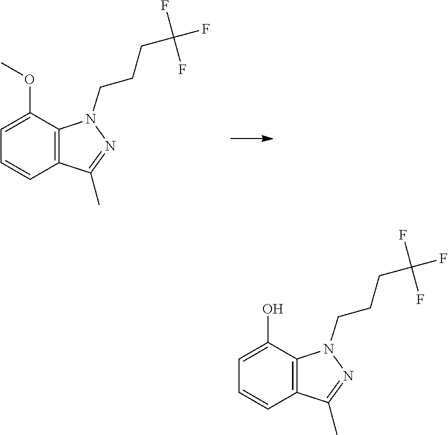 [SMILES: COc1cccc2c(C)nn(CCCC(F)(F)F)c12 ; Cc1nn(CCCC(F)(F)F)c2c(O)cccc12]

To a solution of the crude 7-methoxy-3-methyl-1-(4,4,4-trifluorobutyl)indazole (0.200 g, 0.734 mmol) and 1-dodecanethiol (0.303 g, 1.47 mmol) in DMF (2 mL) was added to which LiO^{t}Bu (1M solution in THF) (1.47 mL, 1.47 mmol). The reaction was heated at 100° C. for 3 hours then allowed to cool to RT. The reaction was quenched with sat. aq. ammonium chloride and extracted with EtOAc. The combined organic extracts were washed with brine, dried over MgSO₄ and evaporated to dryness under reduced pressure. The crude product was purified by flash chromatography on silica gel using a gradient of 0-25% EtOAc/cyclohexane to give the desired product as an inseparable mixture with 1-(4,4,4-trifluorobutyl)indazol-7-ol which was used in the next step without further purification.

### Step 3: Synthesis of 7-(5-chloropyrimidin-2-yl)oxy-3-methyl-1-(4,4,4-trifluorobutyl)indazole (1.021)

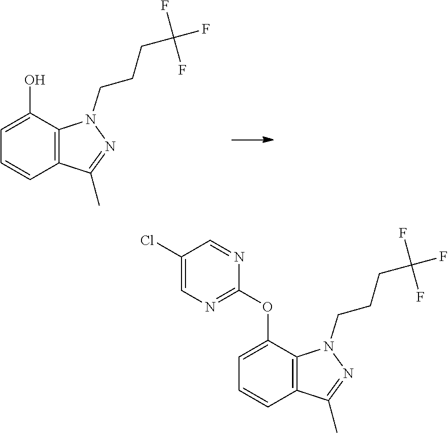 [SMILES: Cc1nn(CCCC(F)(F)F)c2c(O)cccc12 ; Cc1nn(CCCC(F)(F)F)c2c(Oc3ncc(Cl)cn3)cccc12]

To a solution of crude 3-methyl-1-(4,4,4-trifluorobutyl)indazol-7-ol (0.130 g, 0.503 mmol) and K₂CO₃ (0.139 g, 1.01 mmol) in DMF (1.3 mL) was added 2,5-dichloropyrimidine (0.091 g, 0.604 mmol). The reaction was heated at 80° C. for 3 hours the allowed to cool to RT. The reaction was diluted with water and extracted with EtOAc. The combined organic extracts were washed with brine, dried over MgSO₄ and evaporated to dryness under reduced pressure. The crude product was purified by mass-directed reverse phase HPLC to give the desired product (0.116 g, 62%) as a pale brown solid.

¹H NMR (400 MHz, CDCl₃) δ 8.53 (s, 2H), 7.58 (dd, 1H), 7.21-7.14 (m, 2H), 4.42 (t, 2H), 2.59 (s, 3H) 2.09-1.92 (m, 4H).

### EXAMPLE 5: SYNTHESIS OF 7-(5-CHLOROPYRIMIDIN-2-YL)OXY-1-(4,4,4-TRIFLUOROBUTYL)-3-(TRIFLUOROMETHYL)INDAZOLE (1.022)

### Step 1: Synthesis of 7-methoxy-1-(4,4,4-trifluorobutyl)-3-(trifluoromethyl)indazole

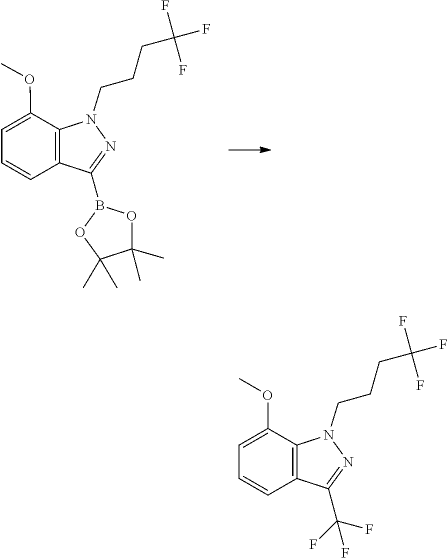 [SMILES: COc1cccc2c(B3OC(C)(C)C(C)(C)O3)nn(CCCC(F)(F)F)c12 ; COc1cccc2c(C(F)(F)F)nn(CCCC(F)(F)F)c12]

A stirred solution of 7-methoxy-3-(4,4,5,5-tetramethyl-1,3,2-dioxaborolan-2-yl)-1-(4,4,4-trifluorobutyl)indazole (0.500 g, 1.30 mmol), KF (0.076 g, 1.30 mmol) and (1,10-phenanthroline)(trifluoromethyl)copper(I) (0.543 g, 1.56 mmol) in DMF (13 mL) was heated at 50° C. for 3 hours and then allowed to cool to RT. The reaction mixture was filtered through a pad of Celite and washed through with EtOAc. The filtrate was evaporated to dryness under reduced pressure. The crude product was purified by flash chromatography on silica gel using a gradient of 0-5% EtOAc/cyclohexane as eluent to give the desired product as an inseparable mixture with 7-methoxy-1-(4,4,4-trifluorobutyl)indazole which was used in the next step without purification.

### Step 2: Synthesis of 1-(4,4,4-trifluorobutyl)-3-(trifluoromethyl)indazol-7-ol

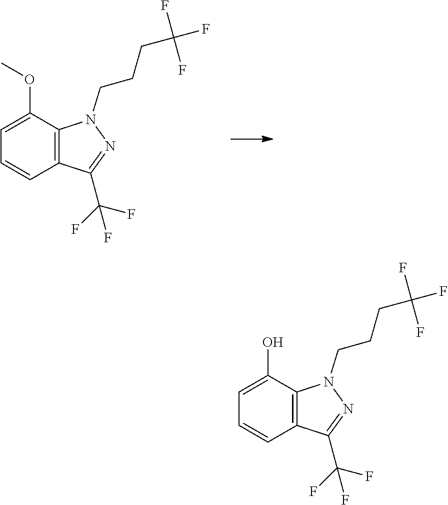 [SMILES: COc1cccc2c(C(F)(F)F)nn(CCCC(F)(F)F)c12 ; Oc1cccc2c(C(F)(F)F)nn(CCCC(F)(F)F)c12]

To a stirred solution of the crude 7-methoxy-1-(4,4,4-trifluorobutyl)-3-(trifluoromethyl)indazole (0.420 g, 1.29 mmol) and 1-dodecanethiol (0.532 g, 2.58 mmol) in DMF (4.2 mL) was added LiO^{t}Bu (1M in THF (2.58 mL, 2.58 mmol). The reaction was stirred at 100° C. for 3 hours and then allowed to cool to RT. The reaction was quenched with sat. aq. ammonium chloride and extracted with EtOAc. The combined organics were washed with brine, dried over MgSO₄ and evaporated to dryness under reduced pressure. The crude product was purified by flash chromatography on silica gel using a gradient of 0-25% EtOAc/cyclohexane as eluent to give the desired product (0.370 g, 92%) as a pale yellow solid.

¹H NMR (400 MHz, CDCl₃) δ 7.39 (d, 1H), 7.09 (t, 1H), 6.74 (d, 1H), 5.59 (s, 1H), 4.78 (t, 2H), 2.29-2.09 (m, 4H).

### Step 3: Synthesis of 7-(5-chloropyrimidin-2-yl)oxy-1-(4,4,4-trifluorobutyl)-3-(trifluoromethyl)indazole (1.022)

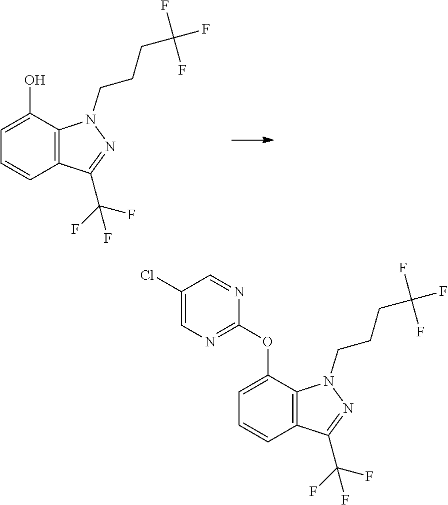 [SMILES: Oc1cccc2c(C(F)(F)F)nn(CCCC(F)(F)F)c12 ; FC(F)(F)CCCn1nc(C(F)(F)F)c2cccc(Oc3ncc(Cl)cn3)c21]

To a stirred solution of 1-(4,4,4-trifluorobutyl)-3-(trifluoromethyl)indazol-7-ol (0.145 g, 0.464 mmol) and K₂CO₃ (0.128 g, 0.929 mmol) in DMF (1.45 mL) was added 2,5-dichloropyrimidine (0.084 g, 0.557 mmol). The reaction was heated at 80° C. for 4 hours, allowed to cool to RT and evaporated to dryness under reduced pressure. The crude product was purified by flash chromatography on silica gel using a gradient of 0-5% EtOAc/cyclohexane as eluent to give the desired product (0.178 g, 90%) as a pale-yellow gum.

¹H NMR (400 MHz, CDCl₃) δ 8.54 (s, 2H), 7.75 (d, 1H), 7.35-7.27 (m, 2H), 4.55 (t, 2H), 2.13-2.04 (m, 4H).

### EXAMPLE 6: SYNTHESIS OF 7-(5-CHLOROPYRIMIDIN-2-YL)OXY-1-(4,4,4-TRIFLUOROBUTYL)BENZOTRIAZOLE (1.020)

### Step 1: Synthesis of 2-methoxy-6-nitro-N-(4,4,4-trifluorobutyl)aniline

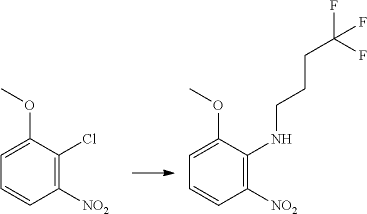 [SMILES: COc1cccc([N+](=O)[O-])c1Cl ; COc1cccc([N+](=O)[O-])c1NCCCC(F)(F)F]

To a solution of 2-chloro-1-methoxy-3-nitro-benzene (0.5 g, 3.0 mmol) in NMP (10 mL) was added N,N-diisopropylethylamine (0.9 mL, 5.0 mmol) and 4,4,4-trifluorobutylamine (0.4 mL, 3.0 mmol). The reaction was heated at 185° C. for 1 hour under microwave irradiation. The reaction was diluted with water (15 mL) and extracted with EtOAc (3×15 mL). The crude product was purified by flash chromatography on silica gel using a gradient of 0-10% EtOAc/cyclohexane as eluent to give the desired product (0.318 g, 40%) as an orange oil.

¹H NMR (400 MHz, CDCl₃) δ 7.74 (d, 1H), 7.62 (br, 1H), 6.94 (d, 1H), 6.68 (t, 1H), 3.86 (s, 3H), 3.61 (q, 2H), 2.25-2.12 (m, 2H), 1.90-1.82 (m, 2H).

### Step 2: Synthesis of 3-methoxy-N2-(4,4,4-trifluorobutyl)benzene-1,2-diamine

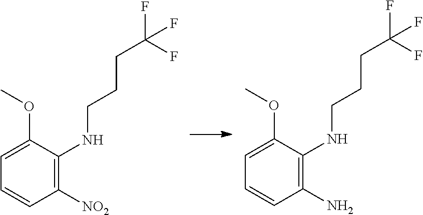 [SMILES: COc1cccc([N+](=O)[O-])c1NCCCC(F)(F)F ; COc1cccc(N)c1NCCCC(F)(F)F]

To a solution of 2-methoxy-6-nitro-N-(4,4,4-trifluorobutyl)aniline (0.318 g, 1.14 mmol) in MeOH (10 mL) was added 5% Pd/C (0.03 g) and the reaction stirred under an atmosphere of H₂ (2 bar pressure) for 45 minutes. The reaction was filtered through Celite, washed through with further MeOH and then evaporated to dryness under reduced pressure to give the desired product (0.283 g, quant) as a red-brown oil which was used without further purification.

¹H NMR (400 MHz, CDCl₃) δ 6.85 (t, 1H), 6.37 (br d, 1H), 6.34 (dd, 1H), 3.80 (s, 3H), 2.98 (t, 2H), 2.32-2.10 (m, 2H), 1.82-1.71 (m, 2H).

### Step 3: Synthesis of 7-methoxy-1-(4,4,4-trifluorobutyl)benzotriazole

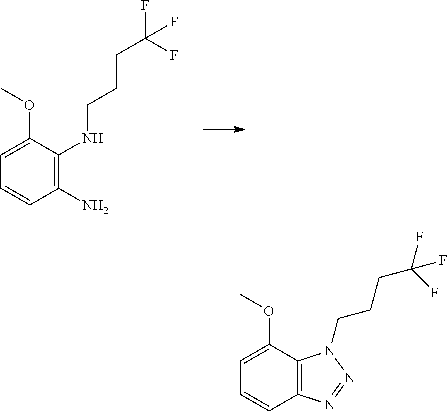 [SMILES: COc1cccc(N)c1NCCCC(F)(F)F ; COc1cccc2nnn(CCCC(F)(F)F)c12]

To a stirred solution of 3-methoxy-N2-(4,4,4-trifluorobutyl)benzene-1,2-diamine (0.283 g, 1.14 mmol) in 2M hydrochloric acid (24 mL) at 0° C. was added a solution of sodium nitrite (0.118 g, 1.71 mmol) in water (1 mL) drop-wise over a few minutes, keeping the reaction temp below 3° C. throughout. The mixture was stirred for 30 minutes, then allowed to warm to room temp. After 1.5 hrs, the stirred reaction mixture was slowly quenched by the addition of saturated NaHCO₃ solution to around pH 7, then extracted with EtOAc (3×15 mL). The combined organic extracts were washed with water (2×10 mL) and evaporated to dryness under reduced pressure to give a red-brown oil. The crude product was purified by column chromatography on silica gel using a gradient of 0-100% EtOAc/cyclohexane to give the product (164 mg, 55%) as an amber oil that crystallised on standing. 1H NMR (400 MHz, CDCl₃) δ 7.62 (d, 1H), 7.27 (dd, 1H), 6.81 (d, 1H), 4.91 (t, 2H), 4.01 (s, 3H), 2.31-2.10 (m, 4H).

### Step 4: Synthesis of 3-(4,4,4-trifluorobutyl)benzotriazol-4-ol

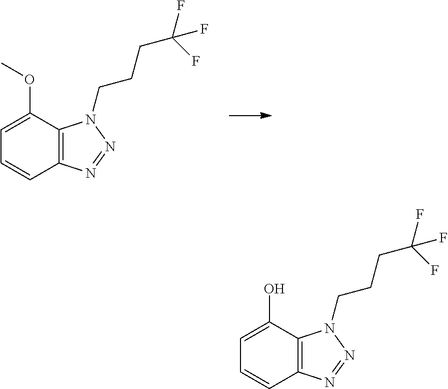 [SMILES: COc1cccc2nnn(CCCC(F)(F)F)c12 ; Oc1cccc2nnn(CCCC(F)(F)F)c12]

To a solution of 7-methoxy-1-(4,4,4-trifluorobutyl)benzotriazole (0.114 g, 0.440 mmol) in DMF (1.2 mL) under a nitrogen atmosphere was added dodecane-1-thiol (0.211 mL, 0.880 mmol) followed by lithium tert-butoxide (1M in THF) (0.880 mL, 0.880 mmol) drop-wise over a few minutes. The mixture was then warmed to 100° C. for 2 hours. The reaction mixture was allowed to cool to room temp, then was quenched by the addition of water (2 mL) followed by 2N HCl to pH6. The mixture was extracted with Et₂O (4×3 mL) and the combined organic extracts evaporated to dryness under reduced pressure. The crude product was purified by column chromatography on silica gel using a gradient of 0-100% EtOAc/cyclohexane to give the product (96 mg, 89%) as a light beige powdery solid.

¹H NMR (400 MHz, CDCl₃) δ 7.62 (d, 1H), 7.19 (dd, 1H), 6.81 (d, 1H), 6.26 (br s, 1H), 4.96 (t, 2H), 2.37-2.25 (m, 2H), 2.25-2.13 (m, 2H).

### Step 5: Synthesis of 7-(5-chloropyrimidin-2-yl)oxy-1-(4,4,4-trifluorobutyl) benzotriazole

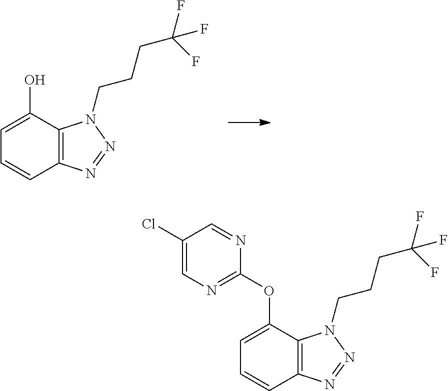 [SMILES: Oc1cccc2nnn(CCCC(F)(F)F)c12 ; FC(F)(F)CCCn1nnc2cccc(Oc3ncc(Cl)cn3)c21]

To a solution of 3-(4,4,4-trifluorobutyl)benzotriazol-4-ol (0.047 g, 0.19 mmol) in DMF (1 mL) at room temp was added K₂CO₃ (0.04 g, 0.29 mmol) followed by 2,5-dichloropyrimidine (0.043 g, 0.29 mmol). The reaction mixture was stirred for 10 minutes then was left to stand at room temp overnight. The reaction mixture was diluted with water (10 mL) and extracted with CH₂Cl₂ (3×8 mL). The combined organic extracts were evaporated to dryness under reduced pressure and the crude material was purified by column chromatography on silica gel using a gradient of 0-100% EtOAc/cyclohexane giving the desired product (62 mg, 91%) as a colourless oil.

¹H NMR (400 MHz, CDCl₃) δ 8.54 (s, 2H), 7.99 (dd, 1H), 7.41 (t, 1H), 7.32 (dd, 1H), 4.71 (t, 2H), 2.21-2.05 (m, 4H).

### EXAMPLE 7: SYNTHESIS OF 5-BROMO-3-CHLORO-7-(5-CHLOROPYRIMIDIN-2-YL)OXY-1-(4.4.4-TRIFLUOROBUTILINDAZOLE (1.010)

### Step 1: Synthesis of 5-bromo-7-methoxy-2-(4,4,4-trifluorobutyl)indazole

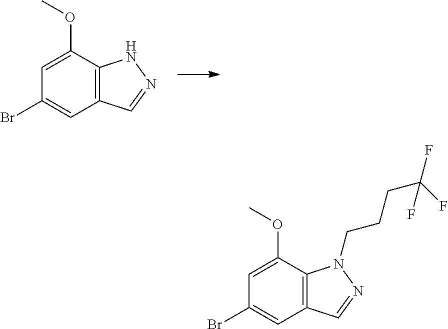 [SMILES: COc1cc(Br)cc2cn[nH]c12 ; COc1cc(Br)cc2cnn(CCCC(F)(F)F)c12]

To a solution of 5-bromo-7-methoxy-1H-indazole (1.00 g, 4.40 mmol) in N,N-dimethylformamide (16 mL) was added 4-bromo-1,1,1-trifluoro-butane (0.865 mL, 7.05 mmol) followed by Cs₂CO₃ (2.55 g, 13.2 mmol). The reaction was heated under microwave irradiation at 80° C. for 1 hour. The reaction mixture was poured into water and diluted with EtOAc. The phases were separated and the aqueous was extracted into EtOAc (×2). The combined organic extracts were washed with brine, dried over MgSO₄ and concentrated in vacuo. The crude material was purified by column chromatography on silica gel using a gradient of 0-70% EtOAc/cyclohexane to give the desired product (1.10 g, 74%) as a yellow solid.

¹H NMR (400 MHz, CDCl₃) δ 7.85 (s, 1H), 7.43 (d, 1H), 6.80 (d, 1H), 4.68 (t, 2H), 3.97 (s, 3H), 2.19-2.02 (m, 4H).

### Step 2: Synthesis of 5-bromo-1-(4,4,4-trifluorobutyl)indazol-7-ol

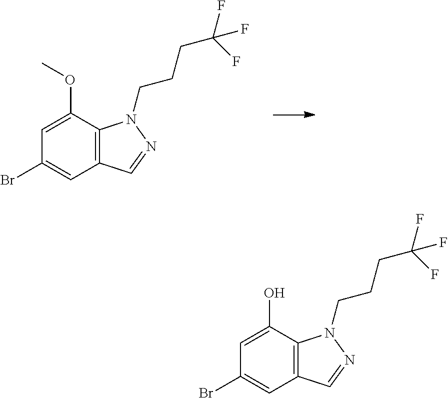 [SMILES: COc1cc(Br)cc2cnn(CCCC(F)(F)F)c12 ; Oc1cc(Br)cc2cnn(CCCC(F)(F)F)c12]

To a solution of 5-bromo-7-methoxy-1-(4,4,4-trifluorobutyl)indazole (0.500 g, 1.48 mmol) in N,N-dimethylformamide (5 mL) at room temperature and under nitrogen was added 1-dodecanethiol (0.725 mL, 2.97 mmol) followed by lithium tert-butoxide (1M in THF) (2.97 mL, 2.97 mmol). On completion of addition, the reaction mixture was heated to 100° C. for 1 h. The reaction mixture was allowed to cool to RT, quenched with water and diluted with EtOAc. The mixture was acidified to pH 1 with 2M HCl and the phases were separated. The aqueous phase was extracted into EtOAc (×2). The combined organic extracts were washed with brine, dried over MgSO₄ and concentrated in vacuo to afford a yellow liquid. The crude product was purified by column chromatography on silica gel using a gradient of 0-60% EtOAc/cyclohexane to give the desired product (460 mg, 96%) as a white solid.

¹H NMR (400 MHz, CDCl₃) δ 7.88 (s, 1H), 7.44 (d, 1H), 6.80 (d, 1H), 6.50 (br s, 1H), 4.72 (t, 2H), 2.23-2.02 (m, 4H).

### Step 3: Synthesis of 5-bromo-7-(5-chloropyrimidin-2-yl)oxy-1-(4,4,4-trifluorobutyl)indazole

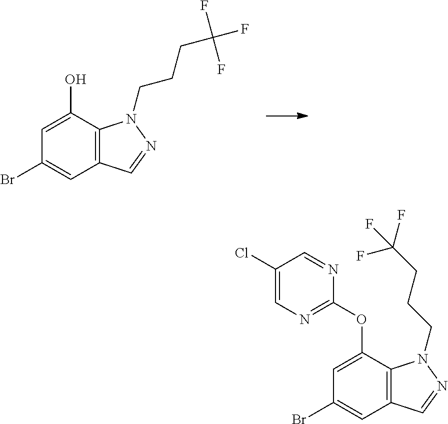 [SMILES: Oc1cc(Br)cc2cnn(CCCC(F)(F)F)c12 ; FC(F)(F)CCCn1ncc2cc(Br)cc(Oc3ncc(Cl)cn3)c21]

To a solution of 5-bromo-1-(4,4,4-trifluorobutyl)indazol-7-ol (0.460 g, 1.42 mmol) in DMF (23.0 mL) was added 2,5-dichloropyrimidine (0.233 g, 1.57 mmol) followed by K₂CO₃ (0.517 g, 3.70 mmol). The reaction mixture was heated to 80° C. for 1 hour. The reaction mixture was allowed to cool to RT, diluted with water and then acidified with 2M HCl. EtOAc was added and the phases were separated, before extracting into EtOAc. The combined organics were washed with brine, dried over MgSO₄, filtered and concentrated to afford a brown liquid. The crude product was purified by column chromatography on silica gel using a gradient of 0-50% EtOAc in cyclohexane to give the desired product (0.6 g, 97%) as a yellow gum which solidified on standing to an off-white solid.

¹H NMR (400 MHz, CDCl₃) δ 8.54 (s, 2H), 7.98 (s, 1H), 7.78 (d, 1H), 7.32 (d, 1H), 4.48 (t, 2H), 2.09-1.97 (m, 4H).

### Step 4: Synthesis of 5-bromo-3-chloro-7-(5-chloropyrimidin-2-yl)oxy-1-(4,4,4-trifluorobutyl)indazole

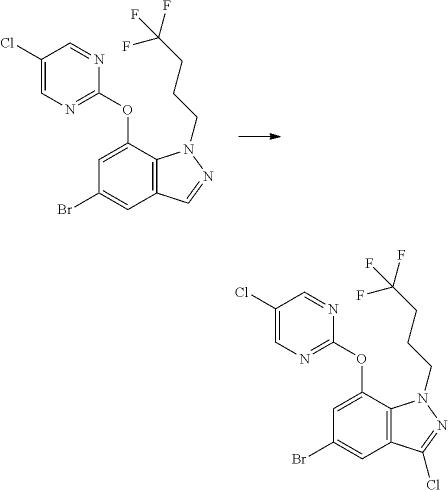 [SMILES: FC(F)(F)CCCn1ncc2cc(Br)cc(Oc3ncc(Cl)cn3)c21 ; FC(F)(F)CCCn1nc(Cl)c2cc(Br)cc(Oc3ncc(Cl)cn3)c21]

A solution of 5-bromo-7-(5-chloropyrimidin-2-yl)oxy-1-(4,4,4-trifluorobutyl)indazole (0.300 g, 0.689 mmol) and N-chlorosuccinimide (0.193 g, 1.45 mmol) in CH₃CN (4.50 mL) was heated under microwave irradiation at 80° C. for 2 hours. The reaction mixture was concentrated in vacuo to afford a yellow gum. The crude product was purified by column chromatography on silica gel using a gradient of 0-30% EtOAc in cyclohexane to give a yellow gum which solidified on standing to an off-white solid which was further purified by mass directed reverse phase HPLC. The desired product (49 mg, 15%) was obtained as a white solid.

¹H NMR (400 MHz, CDCl₃) δ 8.55 (s, 2H), 7.74 (d, 1H), 7.37 (d, 1H), 4.42 (t, 2H), 2.09-1.99 (m, 4H).

### EXAMPLE 8: SYNTHESIS OF 3-CHLORO-7-(5-CHLOROPYRIMIDIN-2-YL)OXY-6-METHYL-1-(4,4,4-TRIFLUOROBUTYL)INDAZOLE (1.043)

### Step 1: Synthesis of 3-chloro-7-(5-chloropyrimidin-2-yl)oxy-6-methyl-1-(4,4,4-trifluorobutyl)indazole (1.043)

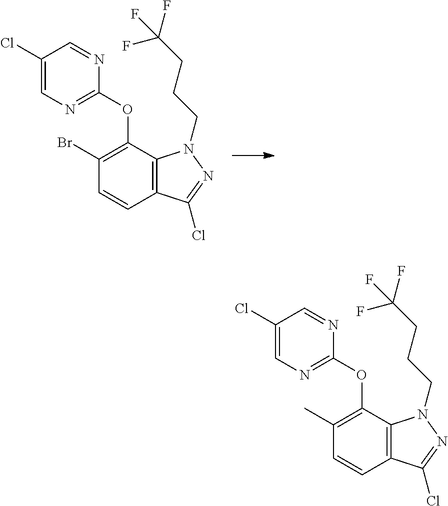 [SMILES: FC(F)(F)CCCn1nc(Cl)c2ccc(Br)c(Oc3ncc(Cl)cn3)c21 ; Cc1ccc2c(Cl)nn(CCCC(F)(F)F)c2c1Oc1ncc(Cl)cn1]

To a solution of 6-bromo-3-chloro-7-(5-chloropyrimidin-2-yl)oxy-1-(4,4,4-trifluorobutyl)indazole (0.100 g, 0.213 mmol), tetrakis(triphenylphosphaniumyl) palladium (0.025 g, 0.021 mmol) and K₂CO₃ (0.148 g, 1.06 mmol) in a mixture of 1,4-dioxane (1.06 mL) and water (0.355 mL) was added trimethylboroxine (0.045 mL, 0.319 mmol). The mixture was heated under microwave irradiation at 140° C. for 1 hour. The reaction mixture was poured into water and diluted with dichloromethane. The aqueous phase was acidified to pH1 with 2M HCl. The phases were separated, and the organic phase was concentrated in vacuo to afford a yellow gum. The crude product was purified by column chromatography on silica gel using a gradient of 0-40% EtOAc in cyclohexane) to give the desired product (45 mg, 51%) as a white solid.

¹H NMR (400 MHz, CDCl₃) δ 8.51 (s, 2H), 7.49 (d, 1H), 7.11 (d, 1H), 4.32 (t, 2H), 2.23 (s, 3H), 2.05-1.93 (m, 4H).

### EXAMPLE 9: SYNTHESIS OF 4-CHLORO-7-(5-CHLOROPYRIMIDIN-2-YL)OXY-1-(4,4,4-TRIFLUOROBUTYL)INDAZOLE (1.013)

### Step 1: Synthesis of 4-chloro-7-methoxy-1-(4,4,4-trifluorobutyl)indazole

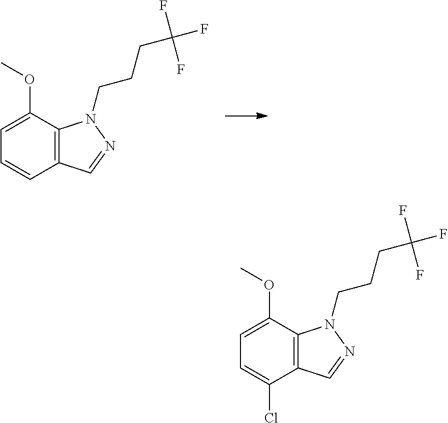 [SMILES: COc1cccc2cnn(CCCC(F)(F)F)c12 ; COc1ccc(Cl)c2cnn(CCCC(F)(F)F)c12]

To a solution of 7-methoxy-1-(4,4,4-trifluorobutyl)indazole (100 mg, 0.39 mmol) in DCM (3 mL) at 0° C. under an atmosphere of nitrogen was added dropwise a solution of sulfuryl chloride (0.032 mL, 0.39 mmol) in 1 mL of DCM. On complete addition the reaction mixture was stirred for 60 mins. The reaction mixture was poured into water and extracted into DCM. The organics were washed with brine, separated, dried over MgSO₄ and concentrated. The crude reaction mixture was purified by column chromatography on silica gel using a gradient of EtOAc in cyclohexane to give the desired product (84 mg, 74% yield) as a colourless oil.

¹H NMR (400 MHz, CDCl₃) δ 7.99 (s, 1H), 6.99 (d, 1H), 6.62 (d, 1H), 4.71 (t, 2H), 3.96 (s, 3H), 2.04-2.30 (m, 4H).

### Step 2: Synthesis of 4-chloro-1-(4,4,4-trifluorobutyl)indazol-7-ol

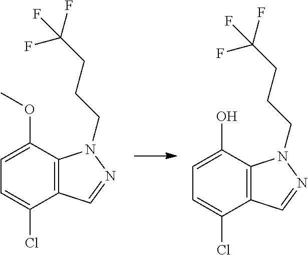 [SMILES: COc1ccc(Cl)c2cnn(CCCC(F)(F)F)c12 ; Oc1ccc(Cl)c2cnn(CCCC(F)(F)F)c12]

To a solution of 4-chloro-7-methoxy-1-(4,4,4-trifluorobutyl)indazole (0.375 g, 1.28 mmol) in DMF (9.38 mL) under an atmosphere of nitrogen was added dodecane-1-thiol (0.583 mL, 2.43 mmol) and lithium tert-butoxide (1M in THF) (2.43 mL, 2.43 mmol). The reaction mixture was heated to 100° C. for 1 hr, allowed to cool to RT then poured into 2M HCl and extracted with EtOAc. The organic extracts were washed with brine, dried over MgSO₄ and concentrated. The crude product was purified by column chromatography on silica gel using a gradient 0-95% EtOAc in cyclohexane to give the desired product (0.291 g, 82% Yield) as a white solid.

¹H NMR (400 MHz, CDCl₃) δ 8.03 (s, 1H), 6.92 (d, 1H), 6.59 (d, 1H), 5.87 (s, 1H), 4.75 (t, 2H), 2.24-2.05 (m, 4H).

### Step 3: Synthesis of 4-chloro-7-(5-chloropyrimidin-2-yl)oxy-1-(4,4,4-trifluorobutyl)indazole

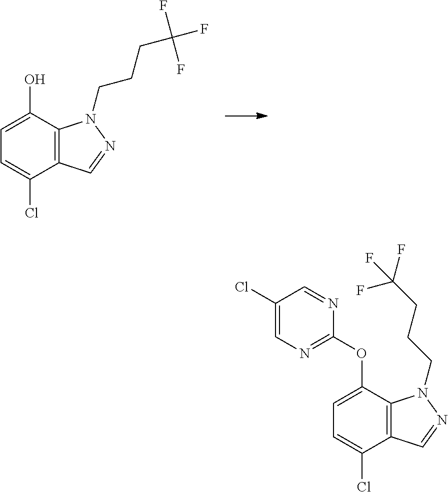 [SMILES: Oc1ccc(Cl)c2cnn(CCCC(F)(F)F)c12 ; FC(F)(F)CCCn1ncc2c(Cl)ccc(Oc3ncc(Cl)cn3)c21]

To a solution of 4-chloro-1-(4,4,4-trifluorobutyl)indazol-7-ol (0.287 g, 1.03 mmol) and 2,5-dichloropyrimidine (0.169 g, 1.13 mmol) in DMF (14.4 mL) was added K₂CO₃ (0.374 g, 2.68 mmol). The reaction was heated to 80° C. for 1 hour, then allowed to cool to RT, diluted with water and then acidified with 2M HCl. EtOAc was added, and the phases were separated, before extracting into EtOAc (×2). The combined organics were washed with brine, dried over MgSO₄, filtered and concentrated. The crude product was purified by column chromatography on silica gel using a gradient of 0-100% EtOAc in cyclohexane to give the desired product (0.344 g, 85%) as a white solid.

¹H NMR (400 MHz, CDCl₃) δ 8.53 (s, 2H), 8.11 (s, 1H), 7.13 (d, 1H), 7.11 (d, 1H), 4.52-4.43 (m, 2H), 2.09-1.97 (m, 4H).

### EXAMPLE 10: SYNTHESIS OF 3-CHLORO-7-(5-CHLOROPYRIMIDIN-2-YL)OXY-1-(4,4,4-TRIFLUOROBUTYL)INDAZOLE-6-CARBONITRILE (1.041)

### Step 1: Synthesis of 3-chloro-7-(5-chloropyrimidin-2-yl)oxy-1-(4,4,4-trifluorobutyl)indazole-6-carbonitrile

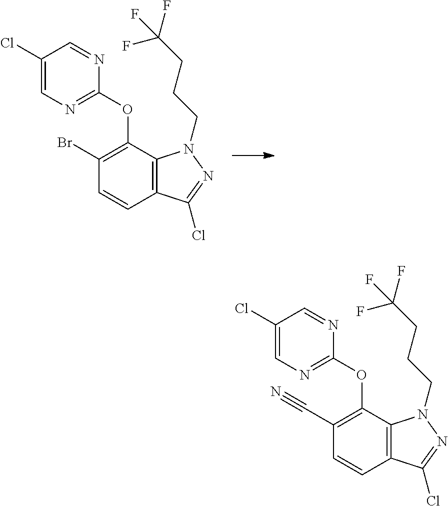 [SMILES: FC(F)(F)CCCn1nc(Cl)c2ccc(Br)c(Oc3ncc(Cl)cn3)c21 ; N#Cc1ccc2c(Cl)nn(CCCC(F)(F)F)c2c1Oc1ncc(Cl)cn1]

To a solution of 6-bromo-3-chloro-7-(5-chloropyrimidin-2-yl)oxy-1-(4,4,4-trifluorobutyl)indazole (0.060 g, 0.13 mmol) and Zn(CN)₂ (0.018 g, 0.15 mmol) in DMF (1.3 mL) was added tetrakis(triphenylphosphine) palladium(0) (0.015 g, 0.013 mmol). The mixture was then heated under microwave irradiation at 125° C. for 90 minutes. The reaction mixture was concentrated and purified by column chromatography on silica gel using a gradient of 0-40% EtOAc in cyclohexane to give the desired product (15 mg, 28%) as a white solid.

¹H NMR (400 MHz, CDCl₃) δ 8.55 (s, 2H), 7.69 (d, 1H), 7.43 (d, 1H), 4.45 (t, 2H), 2.10-1.97 (m, 4H).

### EXAMPLE 11: SYNTHESIS OF 3-BROMO-7-(5-CHLOROPYRIMIDIN-2-YL)OXY-1-(4,4,4-TRIFLUOROBUTYL)INDAZOLE (1.006) AND 7-(5-CHLOROPYRIMIDIN-2-YL)OXY-3-METHOXY-1-(4,4,4-TRIFLUOROBUTYL)INDAZOLE (1.033)

### Step 1: Synthesis of 3-bromo-7-(5-chloropyrimidin-2-yl)oxy-1-(4,4,4-trifluorobutyl)indazole

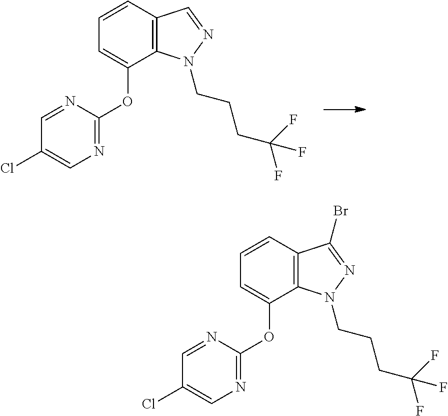 [SMILES: FC(F)(F)CCCn1ncc2cccc(Oc3ncc(Cl)cn3)c21 ; FC(F)(F)CCCn1nc(Br)c2cccc(Oc3ncc(Cl)cn3)c21]

To a solution of 7-(5-chloropyrimidin-2-yl)oxy-1-(4,4,4-trifluorobutyl)indazole (0.530 g, 1.49 mmol) acetonitrile (7.45 mL) was added N-bromosuccinimide (0.588 g, 3.27 mmol). The reaction was heated at 80° C. for 90 minutes under microwave irradiation. The reaction mixture was concentrated and purified by column chromatography on silica gel using a gradient of 0-20% EtOAc/cyclohexane to give the desired product (0.50 g, 77%) as a colourless oil.

¹H NMR (400 MHz, CDCl₃) δ 8.53 (s, 2H), 7.56-7.52 (m, 1H), 7.25-7.21 (m, 2H), 4.45 (br t, 2H), 2.10-1.99 (m, 4H).

### Step 2: Synthesis of 7-(5-chloropyrimidin-2-yl)oxy-3-methoxy-1-(4,4,4-trifluorobutyl)indazole (1.033)

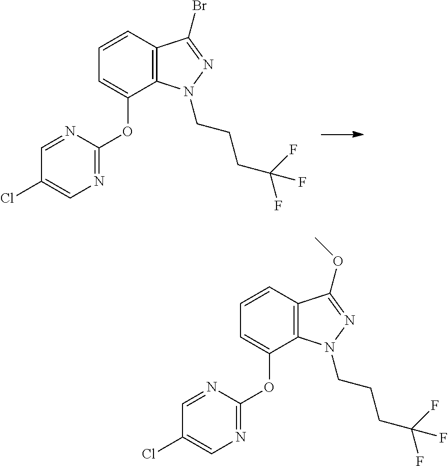 [SMILES: FC(F)(F)CCCn1nc(Br)c2cccc(Oc3ncc(Cl)cn3)c21 ; COc1nn(CCCC(F)(F)F)c2c(Oc3ncc(Cl)cn3)cccc12]

To a solution of 3-bromo-7-(5-chloropyrimidin-2-yl)oxy-1-(4,4,4-trifluorobutyl)indazole (0.100 g, 0.23 mmol), cesium carbonate (0.075 g, 0.23 mmol), Rockphos Pd G3 (0.01 g, 0.01 mmol) and MeOH (0.074 g, 2.30 mmol) in 1,4-dioxane (1 mL) were heated at 90° C. for 1 hour under microwave irradiation. The reaction mixture was diluted with water and extracted with DCM. The organics were dried over MgSO₄, filtered and concentrated under vacuum and purified by mass directed reverse phase HPLC to afford the desired product (0.020 g, 23%) as a brown gum.

¹H NMR (400 MHz, CDCl₃) δ 8.52 (s, 2H), 7.56 (dd, 1H), 7.17-7.12 (m, 1H), 7.09-7.03 (m, 1H), 4.25 (t, 2H). 4.08 (s, 3H), 2.06-1.90 (m, 4H).

### EXAMPLE 12: SYNTHESIS OF 3-CHLORO-7-(5-CHLOROPYRIMIDIN-2-YL)OXY-1-(4,4,4-TRIFLUOROBUTYL)-4-(TRIFLUOROMETHYL)INDAZOLE (1.079)

### Step 1: Synthesis of 1H-indazol-7-yl trifluoromethanesulfonate

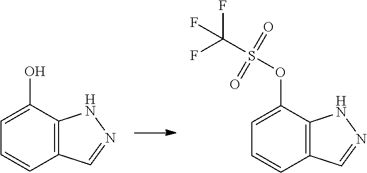 [SMILES: Oc1cccc2cn[nH]c12 ; O=S(=O)(Oc1cccc2cn[nH]c12)C(F)(F)F]

To a solution of 1H-indazol-7-ol (25.0 g, 186 mmol) in THF (500 mL) at 4° C. was added Cs₂CO₃ (60.8 g, 186 mmol). To this was added portion-wise, over 30 minutes 1,1,1-trifluoro-N-(2-pyridyl)-N-(trifluoromethylsulfonyl)methanesulfon-amide (66.8 g, 186 mmol). The mixture was stirred at 5° C. for 2 hours, then, to the reaction mixture at 5° C. was added water (200 mL). The mixture was warmed to 20° C. and the THF was removed in vacuo. The resultant aqueous solution was extracted into EtOAc and the combined organic extracts were washed with brine, dried over magnesium sulfate and concentrated under vacuum. The crude material was purified by column chromatography on silica gel using a gradient of 0-40% EtOAc in cyclohexane to give the desired product (42.87 g, 86%) as a beige solid.

¹H NMR (400 MHz, CDCl₃) δ 10.70 (br s, 1H), 8.20 (s, 1H), 7.80 (d, 1H), 7.36 (d, H), 7.24-7.19 (m, 1H).

### Step 2: Synthesis of (3-chloro-1H-indazol-7-yl) trifluoromethanesulfonate

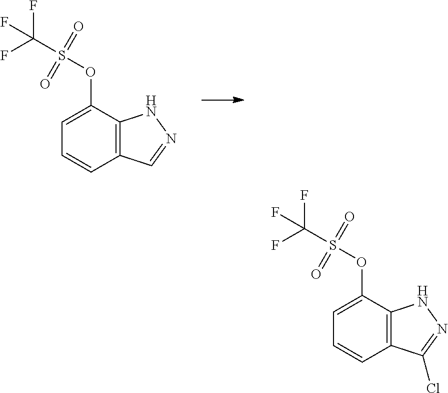 [SMILES: O=S(=O)(Oc1cccc2cn[nH]c12)C(F)(F)F ; O=S(=O)(Oc1cccc2c(Cl)n[nH]c12)C(F)(F)F]

To a stirred solution of 1H-indazol-7-yl trifluoromethanesulfonate (42.8 g, 161 mmol) in DMF (400 mL) at 20° C. was added N-chlorosuccinimide (21.5 g, 161 mmol) portion-wise over 10 minutes. The reaction was heated at 30° C. for 18 hours overnight. The reaction mixture was concentrated afford a yellow liquid and purified by column chromatography on silica gel using a gradient of 0-40% EtOAc in cyclohexane to give the desired product (46.63 g, 97%) as a beige solid.

¹H NMR (400 MHz, CDCl₃) δ 10.50 (br s, 1H), 7.75 (d, 1H), 7.42 (d, 1H), 7.31-7.24 (m, 1H).

### Step 3: Synthesis of [3-chloro-1-(4,4,4-trifluorobutyl)indazol-7-yl] trifluoro methanesulfonate

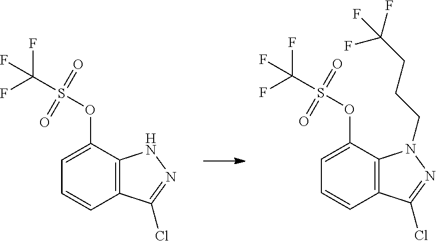 [SMILES: O=S(=O)(Oc1cccc2c(Cl)n[nH]c12)C(F)(F)F ; O=S(=O)(Oc1cccc2c(Cl)nn(CCCC(F)(F)F)c12)C(F)(F)F]

To a stirred solution of (3-chloro-1H-indazol-7-yl) trifluoromethanesulfonate (0.500 g, 1.66 mmol) in THF (5 mL) under an atmosphere of N2 was added (NE)-N-(piperidine-1-carbonylimino)piperidine-1-carboxamide (0.441 g, 1.75 mmol) followed by 4,4,4-trifluorobutan-1-ol (0.185 mL, 1.75 mmol). The reaction was cooled to 0° C. and tributylphosphane (0.50 mL, 2.00 mmol) was added dropwise. After 10 mins, the reaction mixture was allowed to warm to room temperature and stirred overnight. The reaction mixture was quenched by addition of H₂O and diluted with EtOAc, the organic layer was separated and aqueous was re-extracted with EtOAc. The combined organic extracts were washed with saturated aqueous sodium thiosulphate solution then brine, dried over MgSO₄, filtered and concentrated under reduced pressure. The crude material was purified by column chromatography on silica gel using a gradient of 0-20% EtOAc/cyclohexane to give the desired product (0.356 g, 52%) as a colourless oil.

¹H NMR (400 MHz, CDCl₃) δ 7.70 (d, 1H), 7.40 (d, 1H), 7.24 (d, 1H), 4.60 (m, 2H), 2.20 (m, 4H).

### Step 4: Synthesis of [3-chloro-1-(4,4,4-trifluorobutyl)-4-(trifluoromethyl)indazol-7-yl]trifluoromethanesulfonate

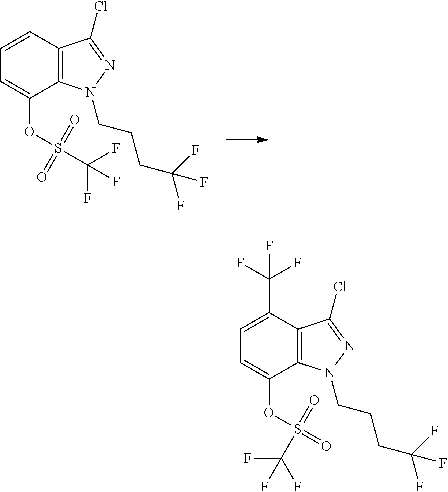 [SMILES: O=S(=O)(Oc1cccc2c(Cl)nn(CCCC(F)(F)F)c12)C(F)(F)F ; O=S(=O)(Oc1ccc(C(F)(F)F)c2c(Cl)nn(CCCC(F)(F)F)c12)C(F)(F)F]

A vial equipped with a stir bar was charged with pyridine-N-oxide (0.29 g, 3.0 mmol), tris(2,2′-bipyridyl)dichlororuthenium(II) hexahydrate (0.0075 g, 0.010 mmol) and [3-chloro-1-(4,4,4-trifluorobutyl)indazol-7-yl] trifluoromethanesulfonate (0.41 g, 1.0 mmol) and acetonitrile (2.5 mL). (2,2,2-Trifluoroacetyl)-2,2,2-trifluoroacetate (0.83 mL, 6.0 mmol) was then added and the reaction irradiated with blue light for 18 hours. The reaction mixture was quenched with saturated aqueous sodium bicarbonate solution and extracted with EtOAc. The combined organic extracts were concentrated under vacuum and purified by column chromatography on silica gel using a gradient of 0-20% EtOAc/cyclohexane to give the desired product (0.229 g, 48%) as a colourless oil.

¹H NMR (400 MHz, CDCl₃) δ 7.63-7.58 (m, 1H), 7.46 (d, 1H), 4.67-4.56 (m, 2H), 2.31-2.16 (m, 4H).

### Step 5: Synthesis of 3-chloro-7-(5-chloropyrimidin-2-yl)oxy-1-(4,4,4-trifluorobutyl)-4-(trifluoromethyl)indazole

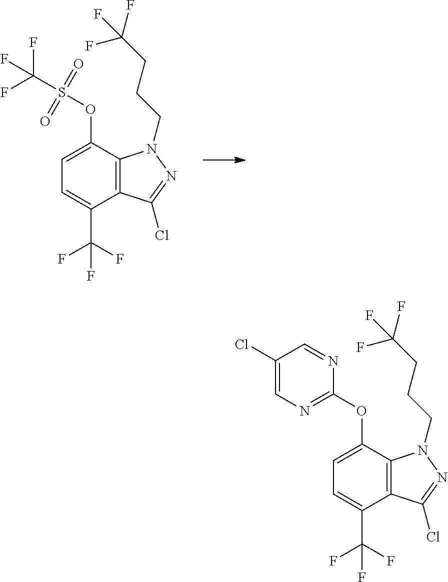 [SMILES: O=S(=O)(Oc1ccc(C(F)(F)F)c2c(Cl)nn(CCCC(F)(F)F)c12)C(F)(F)F ; FC(F)(F)CCCn1nc(Cl)c2c(C(F)(F)F)ccc(Oc3ncc(Cl)cn3)c21]

To a solution of [3-chloro-1-(4,4,4-trifluorobutyl)-4-(trifluoromethyl)indazol-7-yl] trifluoromethanesulfonate (50 mg, 0.104 mmol) in acetonitrile (0.4 mL) was added Cs₂CO₃ (101 mg, 0.522 mmol) followed by 2,5-dichloropyrimidine (23 mg, 0.157 mmol). The reaction mixture was then heated at 80° C. overnight. The crude reaction mixture was quenched with 2M HCl (10 mL) and extracted with EtOAc. The combined organic extracts were concentrated under vacuum and purified by column chromatography on silica gel, using a gradient of 10-20% cyclohexane/EtOAc to give the desired product (21 mg, 44%) as a colourless oil.

¹H NMR (400 MHz, CDCl₃) δ 8.58-8.54 (m, 2H), 7.58 (d, 1H), 7.29 (d, 1H), 4.54 (t, 2H), 2.12-2.03 (m, 4H).

TABLE 1
Examples of herbicidal compounds of the present invention.

| | | ¹H NMR (400 MHz, |
|---|---|---|
| Compound | Structure | CDCl₃ unless stated) |
| 1.001 | 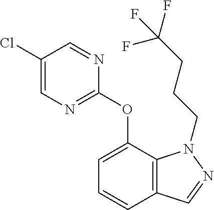 [SMILES: FC(F)(F)CCCn1ncc2cccc(Oc3ncc(Cl)cn3)c21] | 8.53 (s, 2H), 8.05 (s, 1H), 7.68-7.62 (m, 1H), 7.20-7.15 (m, 2H), 4.48 (br t, 2H), 2.10- 198 (m, 2H) |
| 1.002 | 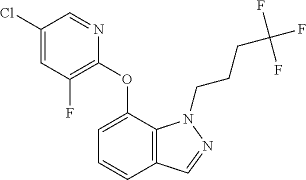 [SMILES: Fc1cc(Cl)cnc1Oc1cccc2cnn(CCCC(F)(F)F)c12] | 8.04 (s, 1H), 7.92 (d, 1H), 7.64-7.58 (m, 2H), 7.15 (s, 1H), 7.13 (d, 1H), 4.54 (t, 2H), 2.14-2.01 (m, 4H) |
| 1.003 | 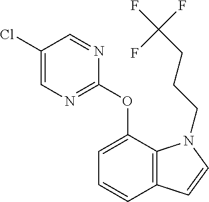 [SMILES: FC(F)(F)CCCn1ccc2cccc(Oc3ncc(Cl)cn3)c21] | 8.51 (s, 2H), 7.53 (s, 1H), 7.11 (t, 1H), 7.02 (d, 1H), 6.96 (dd, 1H), 6.55 (d, 1H), 4.21 (br t, 2H), 2.07-1.92 (m, 4H) |
| 1.004 | 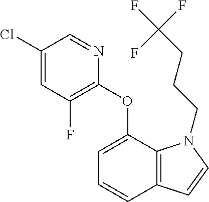 [SMILES: Fc1cc(Cl)cnc1Oc1cccc2ccn(CCCC(F)(F)F)c12] | 7.92 (d, 1H), 7.58 (dd, 1H), 7.49 (dd, 1H), 7.08 (t, 1H), 7.02 (d, 1H), 6.91 (dd, 1H), 6.54 (d, 1H), 4.25 (br t, 2H), 2.11-1.96 (m, 4H) |
| 1.005 | 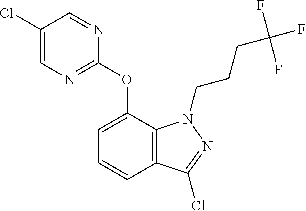 [SMILES: FC(F)(F)CCCn1nc(Cl)c2cccc(Oc3ncc(Cl)cn3)c21] | 8.53 (s, 2H), 7.62-7.57 (m, 1H), 7.25-7.20 (m, 2H), 4.45 (br t, 2H), 2.10-1.99 (m, 4H) |
| 1.006 | 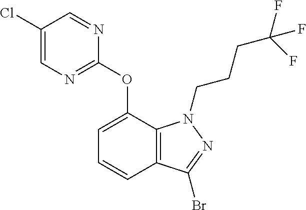 [SMILES: FC(F)(F)CCCn1nc(Br)c2cccc(Oc3ncc(Cl)cn3)c21] | 8.53 (s, 2H), 7.56-7.52 (m, 1H), 7.25-7.21 (m, 2H), 4.45 (br t, 2H), 2.10-1.99 (m, 4H) |
| 1.007 | 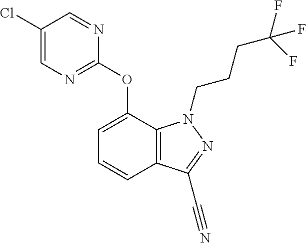 [SMILES: N#Cc1nn(CCCC(F)(F)F)c2c(Oc3ncc(Cl)cn3)cccc12] | 8.54 (s, 2H), 7.76 (dd, 1H), 7.37 (t, 1H), 7.31 (dd, 1H), 4.58 (br t, 2H), 2.17-2.05 (m, 4H) |
| 1.008 | 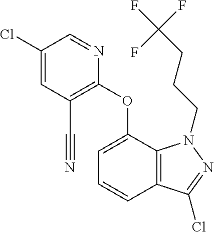 [SMILES: N#Cc1cc(Cl)cnc1Oc1cccc2c(Cl)nn(CCCC(F)(F)F)c12] | 8.26 (d, 1H), 8.07 (d, 1H), 7.61 (dd, 1H), 7.26-7.21 (m, 2H), 4.47 (t, 2H), 2.15-2.03 (m, 4H) |
| 1.009 | 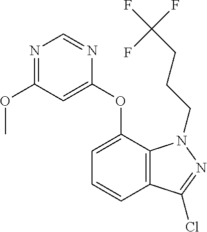 [SMILES: COc1cc(Oc2cccc3c(Cl)nn(CCCC(F)(F)F)c23)ncn1] | 8.45 (s, 1H), 7.58 (dd, 1H), 7.21 (t, 1H), 7.15 (d, 1H), 6.27 (s, 1H), 4.38 (t, 2H), 4.01 (s, 3H), 2.11-1.99 (m, 4H) |
| 1.010 | 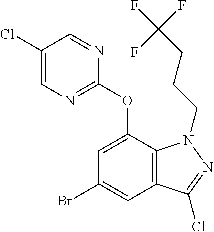 [SMILES: FC(F)(F)CCCn1nc(Cl)c2cc(Br)cc(Oc3ncc(Cl)cn3)c21] | 8.55 (s, 2H), 7.74 (d, 1H), 7.37 (d, 1H), 4.42 (t, 2H), 2.09-1.99 (m, 4H) |
| 1.011 | 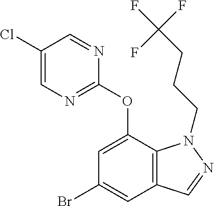 [SMILES: FC(F)(F)CCCn1ncc2cc(Br)cc(Oc3ncc(Cl)cn3)c21] | 8.54 (s, 2H), 7.98 (s, 1H), 7.78 (d, 1H), 7.32 (d, 1H), 4.48 (t, 2H), 2.09-1.97 (m, 4H) |
| 1.012 | 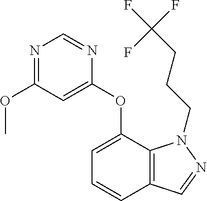 [SMILES: COc1cc(Oc2cccc3cnn(CCCC(F)(F)F)c23)ncn1] | 8.48 (s, 1H), 8.04 (s, 1H), 7.64 (dd, 1H), 7.16 (t, 1H), 7.10 (d, 1H), 6.23 (s, 1H), 4.44 (t, 2H), 4.00 (s, 3H), 2.11-1.97 (m, 4H) |
| 1.013 | 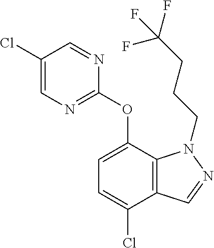 [SMILES: FC(F)(F)CCCn1ncc2c(Cl)ccc(Oc3ncc(Cl)cn3)c21] | 8.53 (s, 2H), 8.11 (s, 1H), 7.12 (q, 2H), 4.48 (br t, 2H), 2.10-1.97 (m, 4H) |
| 1.014 | 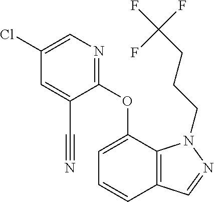 [SMILES: N#Cc1cc(Cl)cnc1Oc1cccc2cnn(CCCC(F)(F)F)c12] 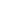 | 8.26 (d, 1H), 8.07 (s, 1H), 8.06 (s, 1H) 7.69-7.63 (m, 1H), 7.18 (s, 1H), 7.17 (d, 1H), 4.53 (t, 2H), 2.15-2.05 (m, 4H) |
| 1.015 | 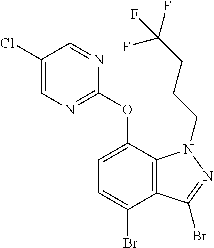 [SMILES: FC(F)(F)CCCn1nc(Br)c2c(Br)ccc(Oc3ncc(Cl)cn3)c21] | 8.53 (s, 2H), 7.37 (d, 1H), 7.07 (d, 1H), 4.46 (t, 2H), 2.10-1.98 (m, 4H) |
| 1.016 | 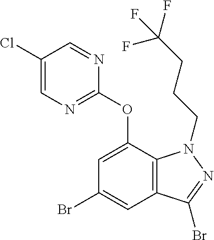 [SMILES: FC(F)(F)CCCn1nc(Br)c2cc(Br)cc(Oc3ncc(Cl)cn3)c21] | 8.55 (s, 2H), 7.68 (d, 1H), 7.38 (d, 1H), 4.44 (t, 2H), 2.11-1.98 (m, 4H) |
| 1.017 | 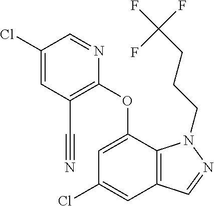 [SMILES: N#Cc1cc(Cl)cnc1Oc1cc(Cl)cc2cnn(CCCC(F)(F)F)c12] | 8.31 (d, 1H), 8.08 (d, 1H), 8.00 (s, 1H), 7.63 (d, 1H), 7.24 (d, 1H), 4.54 (b rt, 2H), 2.13-2.04 (m, 4H) |
| 1.018 | 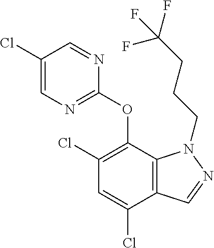 [SMILES: FC(F)(F)CCCn1ncc2c(Cl)cc(Cl)c(Oc3ncc(Cl)cn3)c21] | 8.53 (s, 2H), 8.08 (s, 1H), 7.23 (s, 1H), 4.44 (s, 2H), 2.08-1.94 (m, 4H) |
| 1.019 | 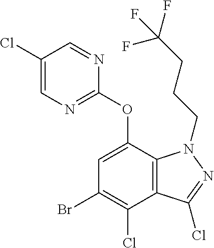 [SMILES: FC(F)(F)CCCn1nc(Cl)c2c(Cl)c(Br)cc(Oc3ncc(Cl)cn3)c21] | 8.55 (s, 2H), 7.46 (s, 1H), 4.42 (t, 2H), 2.10-1.99 (m, 4H) |
| 1.020 | 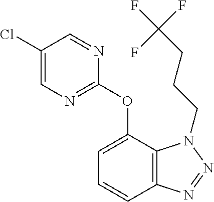 [SMILES: FC(F)(F)CCCn1nnc2cccc(Oc3ncc(Cl)cn3)c21] | 8.54 (s, 2H), 7.99 (dd, 1H), 7.41 (t, 1H), 7.32 (dd, 1H), 4.71 (t, 2H), 2.21-2.05 (m, 4H) |
| 1.021 | 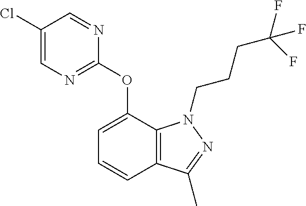 [SMILES: Cc1nn(CCCC(F)(F)F)c2c(Oc3ncc(Cl)cn3)cccc12] | 8.53 (s, 2H), 7.58 (dd, 1H), 7.21-7.14 (m, 2H), 4.42 (t, 2H), 2.59 (s,3H) 2.09-1.92 (m, 4H) |
| 1.022 | 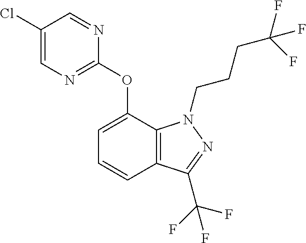 [SMILES: FC(F)(F)CCCn1nc(C(F)(F)F)c2cccc(Oc3ncc(Cl)cn3)c21] | 8.54 (s, 2H), 7.75 (d, 1H), 7.35-7.27 (m, 2H), 4.55 (t, 2H), 2.13-2.04 (m, 4H) |
| 1.023 | 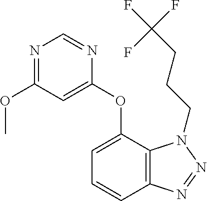 [SMILES: COc1cc(Oc2cccc3nnn(CCCC(F)(F)F)c23)ncn1] | 8.44 (d, 1H), 7.98 (dd, 1H), 7.39 (dd, 1H), 7.23 (dd, 1H), 4.03 (s, 3H), 2.22-2.04 (m, 4H) |
| 1.024 | 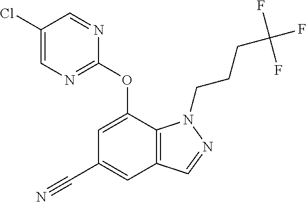 [SMILES: N#Cc1cc(Oc2ncc(Cl)cn2)c2c(cnn2CCCC(F)(F)F)c1] | 8.56 (s, 2H), 8.16 (s, 1H), 8.03 (d, 1H), 7.45 (d, 1H), 4.56 (t, 2H), 2.13-2.01 (m, 4H) |
| 1.025 | 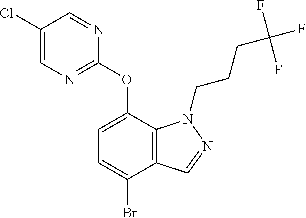 [SMILES: FC(F)(F)CCCn1ncc2c(Br)ccc(Oc3ncc(Cl)cn3)c21] | 8.53 (s, 2H), 8.05 (s, 1H), 7.31 (d, 1H), 7.05 (d, 1H), 4.47 (t, 2H), 2.10-1.97 (m, 4H) |
| 1.026 | 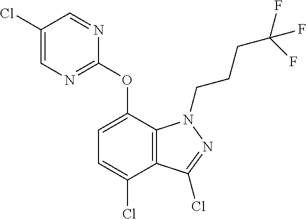 [SMILES: FC(F)(F)CCCn1nc(Cl)c2c(Cl)ccc(Oc3ncc(Cl)cn3)c21] | 8.53 (s, 2H), 7.18-7.11 (m, 2H), 4.42 (t, 2H), 2.10-1.98 (m, 4H) |
| 1.027 | 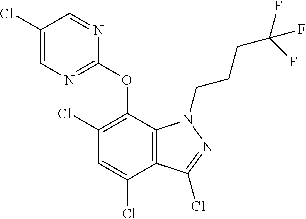 [SMILES: FC(F)(F)CCCn1nc(Cl)c2c(Cl)cc(Cl)c(Oc3ncc(Cl)cn3)c21] | 8.55 (s, 2H), 7.34 (s, 1H), 4.43 (t, 2H), 2.11-1.98 (m, 4H) |
| 1.028 | 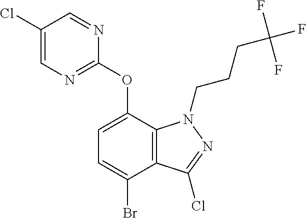 [SMILES: FC(F)(F)CCCn1nc(Cl)c2c(Br)ccc(Oc3ncc(Cl)cn3)c21] | 8.53 (s, 2H), 7.36 (d, 1H), 7.07 (d, 1H), 4.43 (t, 2H), 2.10-1.98 (m, 4H) |
| 1.029 | 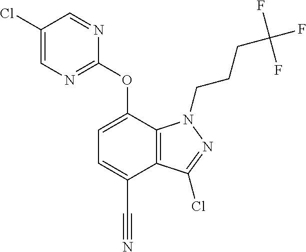 [SMILES: N#Cc1ccc(Oc2ncc(Cl)cn2)c2c1c(Cl)nn2CCCC(F)(F)F] | 8.57 (s, 2H), 7.62 (d, 1H), 7.34 (d, 1H), 4.54 (t, 2H), 2.14-2.03 (m, 4H) |
| 1.030 | 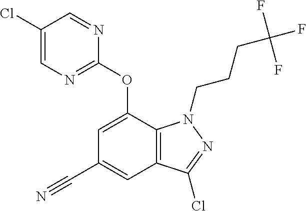 [SMILES: N#Cc1cc(Oc2ncc(Cl)cn2)c2c(c1)c(Cl)nn2CCCC(F)(F)F] | 8.56 (s, 2H), 7.98 (d, 1H), 7.51 (d, 1H), 4.50 (t, 2H), 2.13-2.01 (m, 4H) |
| 1.031 | 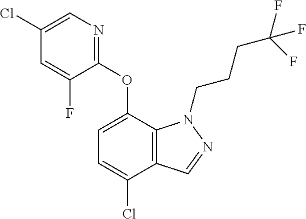 [SMILES: Fc1cc(Cl)cnc1Oc1ccc(Cl)c2cnn(CCCC(F)(F)F)c12] | 8.10 (s, 1H), 7.92 (d, 1H), 7.62 (dd, 1H), 7.10 (d, 2H), 4.53 (t, 2H), 2.15-2.01 (m, 4H) |
| 1.032 | 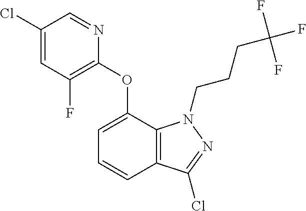 [SMILES: Fc1cc(Cl)cnc1Oc1cccc2c(Cl)nn(CCCC(F)(F)F)c12] | 7.92 (d, 1H), 7.64-7.60 (m, 1H), 7.55 (dd, 1H), 7.22-7.17 (m, 2H), 4.49 (t, 2H), 2.15- 2.02 (m, 4H) |
| 1.033 | 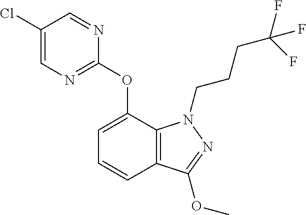 [SMILES: COc1nn(CCCC(F)(F)F)c2c(Oc3ncc(Cl)cn3)cccc12] | 8.52 (s, 2H), 7.56 (dd, 1H), 7.17-7.12 (m, 1H), 7.09- 7.03 (m, 1H), 4.25 (t, 2H), 4.08 (s, 3H), 2.06-1.90 (m, 4H) |
| 1.034 | 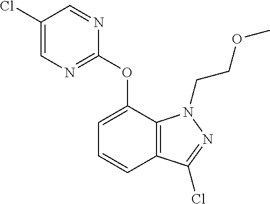 [SMILES: COCCn1nc(Cl)c2cccc(Oc3ncc(Cl)cn3)c21] | 8.51 (s, 2H), 7.61-7.54 (m, 1H), 7.25-7.18 (m, 2H), 4.55 (t, 2H), 3.67 (t, 2H), 3.06 (s, 3H) |
| 1.035 | 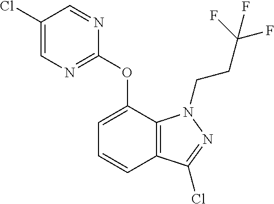 [SMILES: FC(F)(F)CCn1nc(Cl)c2cccc(Oc3ncc(Cl)cn3)c21] | 8.53 (s, 2H), 7.60-7.52 (m, 1H), 7.26-7.20 (m, 2H), 4.65- 4.58 (m, 2H), 2.72-2.60 (m, 2H) |
| 1.036 | 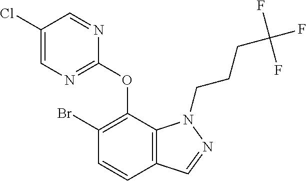 [SMILES: FC(F)(F)CCCn1ncc2ccc(Br)c(Oc3ncc(Cl)cn3)c21] | 8.53 (s, 2H), 8.03 (s, 1H), 7.53 (d, 1H), 7.37 (d, 1H), 4.43 (t, 2H), 2.11-1.95 (m, 4H) |
| 1.037 | 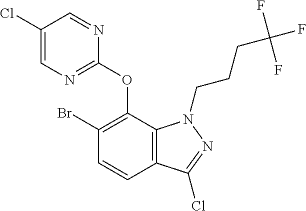 [SMILES: FC(F)(F)CCCn1nc(Cl)c2ccc(Br)c(Oc3ncc(Cl)cn3)c21] | 8.53 (s, 2H), 7.47 (d, 1H), 7.42 (d, 1H), 4.36 (t, 2H), 2.08-1.95 (m, 4H) |
| 1.038 | 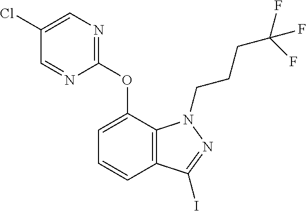 [SMILES: FC(F)(F)CCCn1nc(I)c2cccc(Oc3ncc(Cl)cn3)c21] | 8.52 (s, 2H), 7.44-7.40 (m, 1H), 7.25-7.23 (m, 2H), 4.48 (t, 2H), 2.11-1.99 (m, 4H) |
| 1.039 | 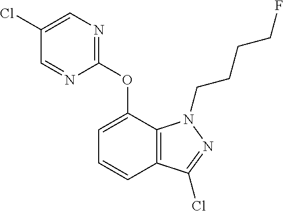 [SMILES: FCCCCn1nc(Cl)c2cccc(Oc3ncc(Cl)cn3)c21] | 8.52 (s, 2H), 7.62-7.55 (m, 1H), 7.24-7.19 (m, 2H), 4.42 (t, 1H), 4.37 (t, 2H), 4.30 (t, 1H), 1.94-1.84 (m, 2H), 1.68- 1.53 (m, 2H) |
| 1.040 | 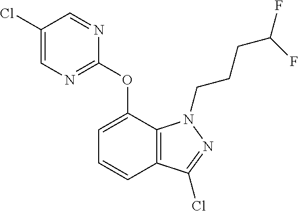 [SMILES: FC(F)CCCn1nc(Cl)c2cccc(Oc3ncc(Cl)cn3)c21] | 8.52 (s, 2H), 7.61-7.57 (m, 1H), 7.22 (dd, 2H), 5.75 tt, 1H), 4.39 (t, 2H), 2.00- 1.89 (m, 2H), 1.86-1.66 (m, 2H) |
| 1.041 | 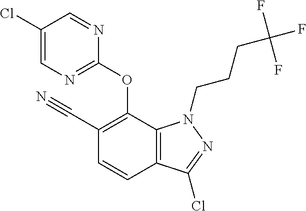 [SMILES: N#Cc1ccc2c(Cl)nn(CCCC(F)(F)F)c2c1Oc1ncc(Cl)cn1] | 8.55 (s, 2H), 7.69 (d, 1H), 7.43 (d, 1H), 4.45 (t, 2H), 2.10-1.97 (m, 4H) |
| 1.042 | 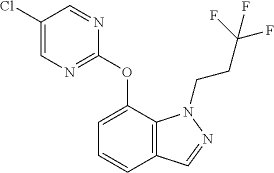 [SMILES: FC(F)(F)CCn1ncc2cccc(Oc3ncc(Cl)cn3)c21] | 8.52 (s, 2H), 8.05 (s, 1H), 7.65-7.60 (m, 1H), 7.20- 7.16 (m, 2H), 4.70-4.62 (m, 2H), 2.72-2.60 (m, 2H) |
| 1.043 | 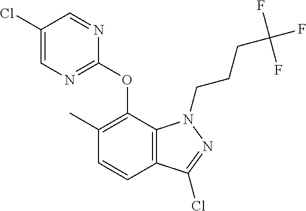 [SMILES: Cc1ccc2c(Cl)nn(CCCC(F)(F)F)c2c1Oc1ncc(Cl)cn1] | 8.51 (s, 2H), 7.49 (d, 1H), 7.11 (d, 1H), 4.32 (t, 2H), 2.23 (s, 3H), 2.05-1.93 (m, 4H) |
| 1.044 | 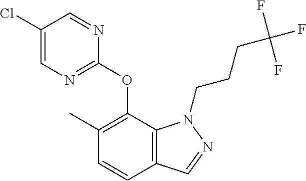 [SMILES: Cc1ccc2cnn(CCCC(F)(F)F)c2c1Oc1ncc(Cl)cn1] | 8.51 (s, 2H), 7.98 (s, 1H), 7.55 (d, 1H), 7.06 (d, 1H), 4.38 (t, 2H), 2.23 (s, 3H), 2.04-1.92 (m, 4H) |
| 1.045 | 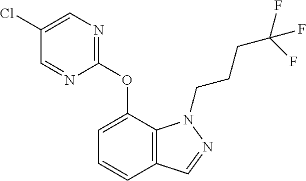 [SMILES: FC(F)(F)CCCn1ncc2cccc(Oc3ncc(Cl)cn3)c21] | 8.91 (br s, 1H), 8.56 (s, 2H), 7.83 (d, 1H), 7.53 (t, 1H), 7.35 (d, 1H), 4.46 (br t, 2H), 2.20-2.15 (m, 4H) |
| 1.046 | 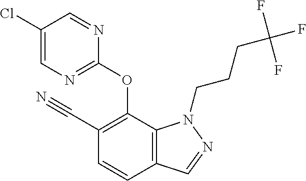 [SMILES: N#Cc1ccc2cnn(CCCC(F)(F)F)c2c1Oc1ncc(Cl)cn1] | 8.55 (s, 2H), 8.13 (s, 1H), 7.73 (d, 1H), 7.38 (d, 1H), 4.51 (t, 2H), 2.09-1.97 (m, 4H) |
| 1.047 | 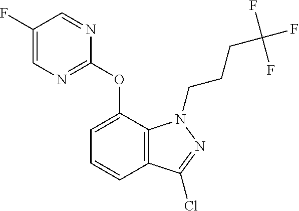 [SMILES: Fc1cnc(Oc2cccc3c(Cl)nn(CCCC(F)(F)F)c23)nc1] | 8.45 (s, 2H), 7.62-7.55 (m, 1H), 7.25-7.21 (m, 2H), 4.44 (t, 2H), 2.10-1.97 (m, 4H) |
| 1.048 | 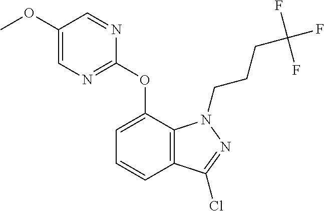 [SMILES: COc1cnc(Oc2cccc3c(Cl)nn(CCCC(F)(F)F)c23)nc1] | 8.25 (s, 2H), 7.59-7.52 (m, 1H), 7.23-7.18 (m, 2H), 4.45 (t, 2H), 3.89 (s, 3H) 2.08- 1.96 (m, 4H) |
| 1.049 | 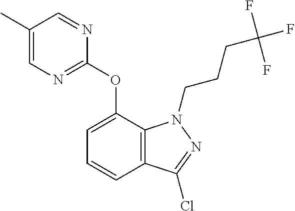 [SMILES: Cc1cnc(Oc2cccc3c(Cl)nn(CCCC(F)(F)F)c23)nc1] | 8.40 (s, 2H), 7.60-7.53 (m, 1H), 7.25-7.20 (m, 2H), 4.43 (t, 2H), 2.29 (s, 3H), 2.07- 1.90 (m, 4H) |
| 1.050 | 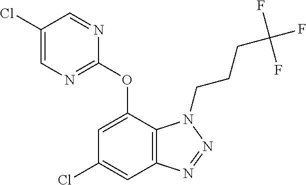 [SMILES: FC(F)(F)CCCn1nnc2cc(Cl)cc(Oc3ncc(Cl)cn3)c21] | 8.56 (s, 2H), 7.97 (s, 1H), 7.38 (s, 1H), 4.70 (t, 2H), 2.21-2.04 (m, 4H) |
| 1.051 | 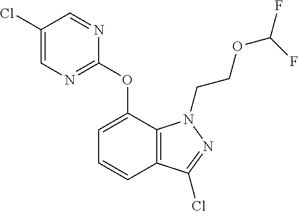 [SMILES: FC(F)OCCn1nc(Cl)c2cccc(Oc3ncc(Cl)cn3)c21] | 8.51 (s, 2H), 7.48 (dd, 1H), 7.25-7.20 (m, 2H), 5.91 (t, 1H), 4.65 (t, 2H), 4.15 (t, 2H) |
| 1.052 | 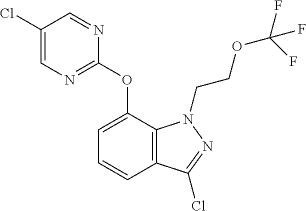 [SMILES: FC(F)(F)OCCn1nc(Cl)c2cccc(Oc3ncc(Cl)cn3)c21] | 8.50 (s, 2H), 7.59 (d, 1H), 7.25-7.21 (m, 2H), 4.72 (t, 2H), 4.30 (t, 2H) |
| 1.054 | 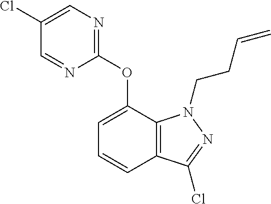 [SMILES: C=CCCn1nc(Cl)c2cccc(Oc3ncc(Cl)cn3)c21] | 8.52 (s, 2H), 7.59-7.55 (m, 1H), 7.22-7.18 (m, 2H), 5.70- 5.58 (m, 1H), 4.92-4.88 (m, 2H), 4.38 (t, 2H), 2.51 (q, 2H) |
| 1.055 | 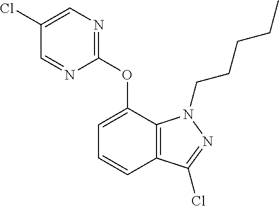 [SMILES: CCCCCn1nc(Cl)c2cccc(Oc3ncc(Cl)cn3)c21] | 8.52 (s, 2H), 7.58-7.55 (m, 1H), 7.20-7.15 (m, 2H), 4.30 (t, 2H), 1.78-1.70 (m, 2H), 1.23-1.10 (m, 4H), 0.78 (t, 3H) |
| 1.056 | 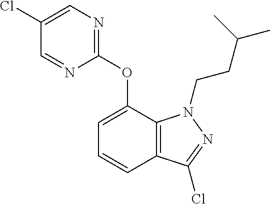 [SMILES: CC(C)CCn1nc(Cl)c2cccc(Oc3ncc(Cl)cn3)c21] | 8.52 (s, 2H), 7.58 (dd, 1H), 7.20-7.15 (m, 2H), 4.30 (t, 2H), 1.67-1.60 (m, 2H), 1.52- 1.45 (m, 1H), 0.83 (d, 6H) |
| 1.057 | 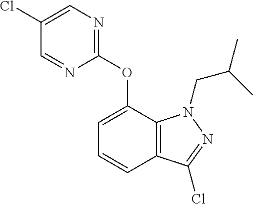 [SMILES: CC(C)Cn1nc(Cl)c2cccc(Oc3ncc(Cl)cn3)c21] | 8.52 (s, 2H), 7.59-7.55 (m, 1H), 7.20-7.17 (m, 2H), 4.11 (d, 2H), 2.22-2.12 (m, 1H), 0.80 (d, 6H) |
| 1.058 | 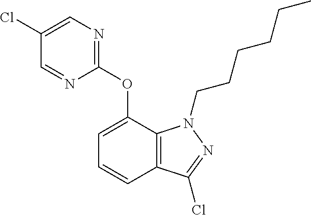 [SMILES: CCCCCCn1nc(Cl)c2cccc(Oc3ncc(Cl)cn3)c21] | 8.52 (s, 2H), 7.58-7.55 (m, 1H), 7.20-7.15 (m, 2H), 4.30 (t, 2H), 1.75-1.70 (m, 2H), 1.21-1.10 (m, 6H), 0.80 (t, 3H) |
| 1.059 | 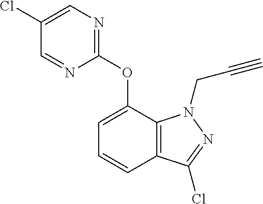 [SMILES: C#CCn1nc(Cl)c2cccc(Oc3ncc(Cl)cn3)c21] | 8.52 (s, 2H), 7.60 (d, 1H), 7.28-7.25 (m, 2H), 5.19 (s, 2H), 2.08 (t, 1H) |
| 1.060 | 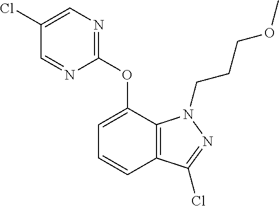 [SMILES: COCCCn1nc(Cl)c2cccc(Oc3ncc(Cl)cn3)c21] | 8.52 (s, 2H), 7.58-7.55 (m, 1H), 7.20-7.16 (m, 2H), 4.41 (t, 2H), 3.27 (t, 2H), 3.19 (s, 3H), 2.05-1.99 (m, 2H) |
| 1.061 | 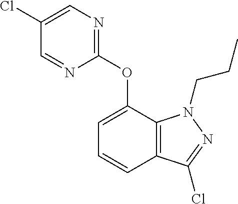 [SMILES: CCCn1nc(Cl)c2cccc(Oc3ncc(Cl)cn3)c21] | 8.52 (s, 2H), 7.60-7.55 (m, 1H), 7.20-7.15 (m, 2H), 4.26 (t, 2H), 1.81-1.75 (m, 2H), 0.78 (t, 3H) |
| 1.062 | 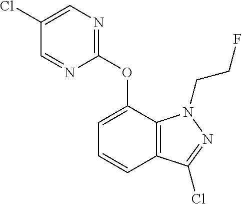 [SMILES: FCCn1nc(Cl)c2cccc(Oc3ncc(Cl)cn3)c21] | (CD₃CN) 8.60 (s, 2H), 7.63 (dd, 1H), 7.34 (dd, 1H), 7.31 (t, 1H), 4.77 (t, 1H), 4.70- 4.63 (m, 2H), 4.63-4.58 (m, 1H) |
| 1.063 | 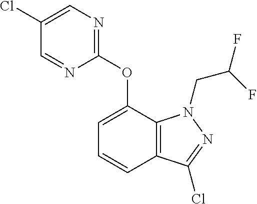 [SMILES: FC(F)Cn1nc(Cl)c2cccc(Oc3ncc(Cl)cn3)c21] | 8.53 (s, 2H), 7.60 (d, 1H), 7.31-7.25 (m, 2H), 6.08 (tt, 1H), 4.78 (td, 2H) |
| 1.064 | 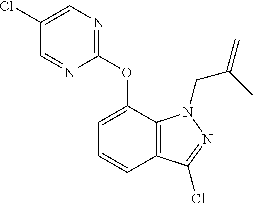 [SMILES: C=C(C)Cn1nc(Cl)c2cccc(Oc3ncc(Cl)cn3)c21] | 8.48 (s, 2H), 7.61-7.55 (m, 1H), 7.24-7.20 (m, 2H), 4.93 (s, 2H), 4.63 (s, 1H), 4.35 (s, 1H), 1.55 (s, 3H) |
| 1.065 | 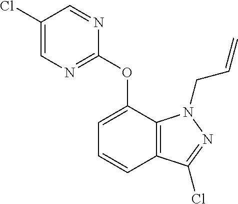 [SMILES: C=CCn1nc(Cl)c2cccc(Oc3ncc(Cl)cn3)c21] | 8.49 (s, 2H), 7.61-7.55 (m, 1H), 7.23-7.18 (m, 2H), 5.87- 5.80 (m, 1H), 5.00-4.88 (m, 4H) |
| 1.066 | 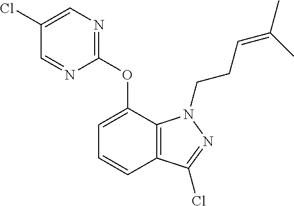 [SMILES: CC(C)=CCCn1nc(Cl)c2cccc(Oc3ncc(Cl)cn3)c21] | 8.51 (s, 2H), 7.58-7.52 (m, 1H), 7.20-7.16 (m, 2H), 4.92 (t, 1H), 4.30 (t, 2H), 2.45 (q, 2H), 1.55 (s, 3H), 1.40 (s, 3H) |
| 1.067 | 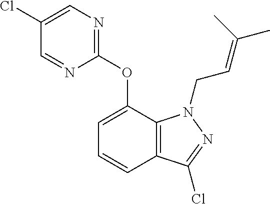 [SMILES: CC(C)=CCn1nc(Cl)c2cccc(Oc3ncc(Cl)cn3)c21] | 8.50 (s, 2H), 7.48 (dd, 1H), 7.20-7.15 (m, 2H), 5.15 (t, 1H), 4.94 (d, 2H), 1.63 (s, 3H), 1.55 (s, 3H) |
| 1.068 | 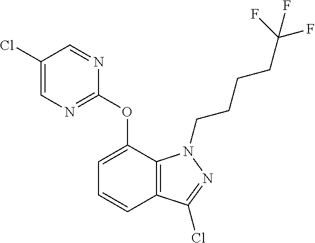 [SMILES: FC(F)(F)CCCCn1nc(Cl)c2cccc(Oc3ncc(Cl)cn3)c21] | 8.51 (s, 2H), 7.59-7.55 (m, 1H), 7.22-7.19 (m, 2H), 4.35 (t, 2H), 2.08-1.97 (m, 2H), 1.88-1.82 (m, 2H), 1.45-1.40 (m, 2H) |
| 1.069 | 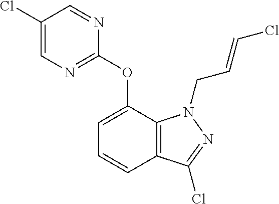 [SMILES: Cl/C=C/Cn1nc(Cl)c2cccc(Oc3ncc(Cl)cn3)c21] | 8.51 (s, 2H), 7.60-7.56 (m, 1H), 7.26-7.21 (m, 2H), 6.00- 5.90 (m, 2H), 4.99 (d, 2H) |
| 1.070 | 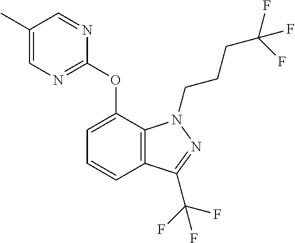 [SMILES: Cc1cnc(Oc2cccc3c(C(F)(F)F)nn(CCCC(F)(F)F)c23)nc1] | 8.41 (s, 2H), 7.73 (d, 1H), 7.34-7.26 (m, 2H), 4.56 (t, 2H), 2.30 (s, 3H), 2.11- 1.95 (m, 4H) |
| 1.071 | 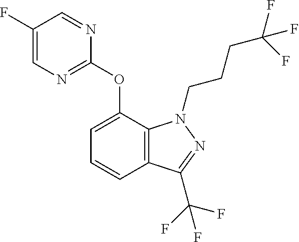 [SMILES: Fc1cnc(Oc2cccc3c(C(F)(F)F)nn(CCCC(F)(F)F)c23)nc1] | 8.47 (s, 2H), 7.75 (dt, 1H), 7.35-7.27 (m, 2H), 4.57 (t, 2H), 2.25-2.02 (m, 4H) |
| 1.072 | 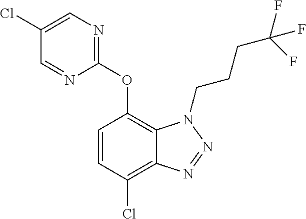 [SMILES: FC(F)(F)CCCn1nnc2c(Cl)ccc(Oc3ncc(Cl)cn3)c21] | 8.53 (s, 2H), 7.41 (d, 1H), 7.29 (d, 1H), 4.70 (t, 2H), 2.20-2.04 (m, 4H) |
| 1.073 | 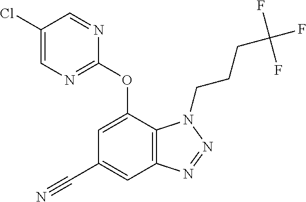 [SMILES: N#Cc1cc(Oc2ncc(Cl)cn2)c2c(c1)nnn2CCCC(F)(F)F] | 8.59 (s, 2H), 8.32 (s, 1H), 7.65 (s, 1H), 4.80 (t, 2H), 2.26-2.08 (m, 4H) |
| 1.074 | 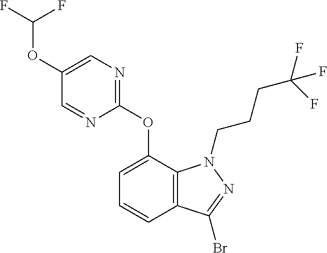 [SMILES: FC(F)Oc1cnc(Oc2cccc3c(Br)nn(CCCC(F)(F)F)c23)nc1] | 8.48 (s, 2H), 7.56- 7.52 (m, 1H), 7.28-7.24 (m, 2H), 6.57 (t, 1H), 4.48 (t, 2H), 2.10-1.95 (m, 4H) |
| 1.075 | 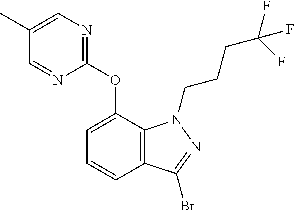 [SMILES: Cc1cnc(Oc2cccc3c(Br)nn(CCCC(F)(F)F)c23)nc1] | 8.40 (s, 2H), 7.54- 7.48 (m, 1H), 7.23-7.20 (m, 2H), 4.45 (t, 2H), 2.30 (s, 3H), 2.05-1.92 (m, 4H) |
| 1.076 | 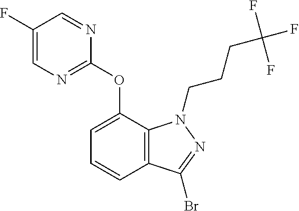 [SMILES: Fc1cnc(Oc2cccc3c(Br)nn(CCCC(F)(F)F)c23)nc1] | 8.43 (s, 2H), 7.56-7.51 (m, 1H), 7.25-7.20 (m, 2H), 4.47 (t, 2H), 2.10-2.00 (m, 4H) |
| 1.077 | 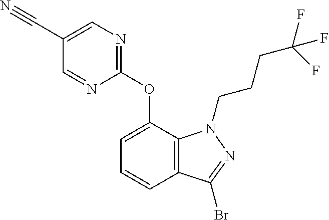 [SMILES: N#Cc1cnc(Oc2cccc3c(Br)nn(CCCC(F)(F)F)c23)nc1] | 8.85 (s, 2H), 7.60-7.55 (m, 1H), 7.28-7.23 (m, 2H), 4.42 (t, 2H), 2.11-2.00 (m, 4H) |
| 1.078 | 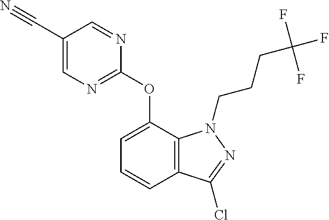 [SMILES: N#Cc1cnc(Oc2cccc3c(Cl)nn(CCCC(F)(F)F)c23)nc1] | 8.86 (s, 2H), 7.66-7.60 (m, 1H), 7.27-7.23 (m, 2H), 4.40 (t, 1H), 2.12-2.00 (m, 4H) |
| 1.079 | 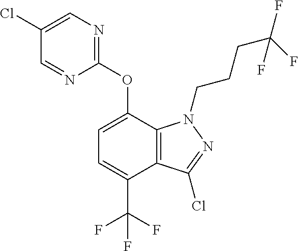 [SMILES: FC(F)(F)CCCn1nc(Cl)c2c(C(F)(F)F)ccc(Oc3ncc(Cl)cn3)c21] | 8.58-8.54 (m, 2H), 7.58 (d, 1H), 7.29 (d, 1H), 4.54 (t, 2H), 2.12-2.03 (m, 4H) |
| 1.080 | 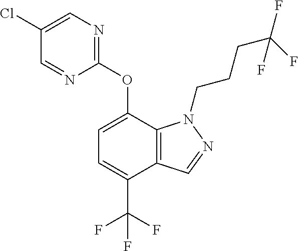 [SMILES: FC(F)(F)CCCn1ncc2c(C(F)(F)F)ccc(Oc3ncc(Cl)cn3)c21] | 8.49 (s, 2H), 8.11 (d, 1H), 7.41 (d, 1H), 7.19 (d, 1H), 4.49 (t, 2H), 2.05-1.92 (m, 4H) |
| 1.081 | 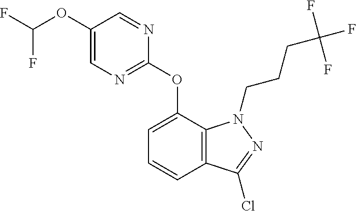 [SMILES: FC(F)Oc1cnc(Oc2cccc3c(Cl)nn(CCCC(F)(F)F)c23)nc1] | 8.47 (s, 2H), 7.61- 7.55 (m, 1H), 7.29-7.21 (m, 2H), 6.57 (t, 1H), 4.48- 4.40 (m, 2H), 2.12-1.95 (m, 4H) |
| 1.082 | 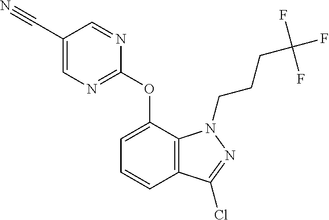 [SMILES: N#Cc1cnc(Oc2cccc3c(Cl)nn(CCCC(F)(F)F)c23)nc1] | 8.86 (s, 2H), 7.66-7.60 (m, 1H), 7.27-7.22 (m, 2H), 4.41- 4.37 (m, 2H), 2.14-1.98 (m, 4H) |
| 1.083 | 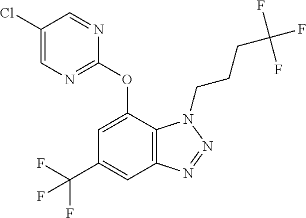 [SMILES: FC(F)(F)CCCn1nnc2cc(C(F)(F)F)cc(Oc3ncc(Cl)cn3)c21] | 8.58 (s, 2H), 8.29 (s, 1H), 7.61 (s, 1H), 4.76 (t, 2H), 2.22-2.06 (m, 4H) |

### BIOLOGICAL EXAMPLES

Seeds of a variety of test species are sown in standard soil in pots Amaranthus retoflexus (AMARE), Echinochloa crus-galli (ECHCG), Setaria faberi (SETFA)). After cultivation for one day (pre-emergence) or after 8 days cultivation (post-emergence) under controlled conditions in a glasshouse (at 24/16° C., day/night; 14 hours light; 65% humidity), the plants are sprayed with an aqueous spray solution derived from the formulation of the technical active ingredient in acetone/water (50:50) solution containing 0.5% Tween™ 20 (polyoxyethelyene sorbitan monolaurate, CAS RN 9005-64-5). Compounds are applied at 250 g/ha unless otherwise stated. The test plants are then grown in a glasshouse under controlled conditions in a glasshouse (at 24/16° C., day/night; 14 hours light; 65% humidity) and watered twice daily. After 13 days for pre- and post-emergence, the test is evaluated for the percentage damage caused to the plant. The biological activities are shown in the following table on a five-point scale (5=81-100%; 4=61-80%; 3=41-60%; 2=21-40%; 1=0-20%).

TABLE B1
Post-emergence Test

| Compound | AMARE | ABUTH | SETFA | ECHCG | IPOHE |
|---|---|---|---|---|---|
| 1.001 | 2 | 4 | 4 | 3 | 1 |
| 1.002 | 2 | 2 | 3 | 3 | 1 |
| 1.003 | 3 | 2 | 1 | 1 | 2 |
| 1.004 | 3 | 1 | 1 | 1 | 2 |
| 1.005 | 5 | 5 | 5 | 5 | 3 |
| 1.006 | 5 | 5 | 4 | 5 | 2 |
| 1.007 | 5 | 5 | 5 | 5 | 4 |
| 1.008 | 5 | 4 | 4 | 4 | 2 |
| 1.010 | 4 | 1 | 2 | 2 | 1 |
| 1.011 | 3 | 1 | 1 | 1 | 1 |
| 1.012 | 1 | 1 | 1 | 1 | 1 |
| 1.013 | 5 | 5 | 5 | 5 | 2 |
| 1.014 | 4 | 4 | 3 | 2 | 2 |
| 1.015 | 4 | 4 | 2 | 2 | 1 |
| 1.016 | 4 | 2 | 1 | 1 | 1 |
| 1.017 | 4 | 1 | 1 | 1 | 1 |
| 1.018 | 1 | 1 | 1 | 1 | 1 |
| 1.019 | 4 | 1 | 2 | 2 | 1 |
| 1.020 | 2 | 3 | 5 | NT | 2 |
| 1.021 | 1 | 4 | 4 | NT | 3 |
| 1.022 | 5 | 5 | 5 | NT | 4 |
| 1.023 | 1 | 1 | 1 | NT | 1 |
| 1.024 | 2 | 2 | 1 | NT | 1 |
| 1.025 | 2 | 2 | 2 | NT | 1 |
| 1.026 | 5 | 4 | 5 | NT | 2 |
| 1.027 | 4 | 2 | 3 | 4 | 1 |
| 1.028 | 5 | 2 | 3 | NT | 1 |
| 1.029 | 4 | 1 | 1 | NT | 1 |
| 1.030 | 4 | 1 | 2 | 2 | 1 |
| 1.031 | 4 | 1 | 1 | 1 | 1 |
| 1.032 | 5 | 3 | NT | 3 | 3 |
| 1.033 | 1 | 1 | 1 | 1 | 1 |
| 1.034 | 5 | 4 | 4 | 4 | 4 |
| 1.035 | 5 | 5 | 4 | 5 | 4 |
| 1.036 | 1 | 1 | 1 | 1 | 1 |
| 1.037 | 2 | 1 | 1 | 1 | 1 |
| 1.039 | 5 | 4 | 4 | 4 | 4 |
| 1.040 | 5 | 4 | 4 | 5 | 5 |
| 1.041 | 1 | 1 | 1 | 1 | 1 |
| 1.042 | 4 | 4 | 4 | 4 | 2 |
| 1.043 | 4 | 3 | 3 | 3 | 4 |
| 1.044 | 1 | 1 | 1 | 1 | 1 |
| 1.046 | 2 | 2 | 1 | 1 | 1 |
| 1.047 | 5 | 5 | 4 | 5 | 5 |
| 1.048 | 5 | 4 | 3 | 3 | 4 |
| 1.049 | 4 | 5 | 4 | 5 | 3 |
| 1.050 | 5 | 2 | 1 | 1 | 3 |
| 1.051 | 5 | 4 | 4 | 4 | 3 |
| 1.052 | 5 | 5 | 3 | 4 | 3 |
| 1.054 | 4 | 4 | 2 | 3 | 2 |
| 1.055 | 4 | 3 | 3 | 3 | 2 |
| 1.056 | 4 | 3 | 2 | 2 | 3 |
| 1.057 | 3 | 2 | 1 | 1 | 1 |
| 1.058 | 4 | 3 | 2 | 3 | 2 |
| 1.059 | 4 | 2 | 1 | 1 | 1 |
| 1.060 | 3 | 3 | 3 | 4 | 2 |
| 1.061 | 4 | 2 | 2 | 2 | 2 |
| 1.062 | 3 | 2 | 1 | 1 | 1 |
| 1.063 | 4 | 3 | 2 | 2 | 2 |
| 1.064 | 2 | 2 | 1 | 1 | 1 |
| 1.065 | 4 | 2 | 2 | 2 | 2 |
| 1.066 | 4 | 4 | 3 | 3 | 2 |
| 1.067 | 4 | 3 | 3 | 3 | 2 |
| 1.068 | 5 | 4 | 3 | 3 | 2 |
| 1.069 | 5 | 4 | 2 | 3 | 2 |
| 1.070 | 3 | 5 | 3 | 4 | 3 |
| 1.071 | 5 | 5 | 4 | 4 | 4 |
| 1.072 | 4 | 1 | 3 | 3 | 3 |

NT = Not Tested.

TABLE B2
Pre-emergence Test

| Compound | AMARE | ABUTH | SETFA | ECHCG | IPOHE |
|---|---|---|---|---|---|
| 1.001 | 4 | 4 | 5 | 5 | 1 |
| 1.002 | 1 | 1 | 2 | 2 | 1 |
| 1.003 | 1 | 1 | 1 | 1 | 1 |
| 1.004 | 1 | 1 | 2 | 1 | 1 |
| 1.005 | 5 | 3 | 5 | 5 | 1 |
| 1.006 | 5 | 2 | 5 | 5 | 1 |
| 1.007 | 5 | 3 | 5 | 5 | 2 |
| 1.008 | 4 | 2 | 4 | 4 | 1 |
| 1.010 | 4 | 1 | 2 | 2 | 1 |
| 1.011 | 1 | 1 | 1 | 1 | 1 |
| 1.012 | 2 | 1 | 1 | 1 | 1 |
| 1.013 | 5 | 2 | 5 | 5 | 1 |
| 1.014 | 4 | 1 | 4 | 4 | 1 |
| 1.015 | 2 | 1 | 1 | 1 | 1 |
| 1.016 | 1 | 1 | 1 | 1 | 1 |
| 1.017 | 1 | 1 | 1 | 1 | 1 |
| 1.018 | NT | 1 | 1 | 1 | 1 |
| 1.019 | 1 | 1 | 1 | 1 | 1 |
| 1.020 | 5 | 4 | 5 | NT | 2 |
| 1.021 | 5 | 3 | 5 | NT | 2 |
| 1.022 | 5 | 4 | 5 | 5 | 4 |
| 1.023 | 1 | 1 | 1 | NT | 1 |
| 1.024 | 4 | 1 | 4 | NT | 1 |
| 1.025 | 1 | 1 | 2 | NT | 1 |
| 1.026 | 5 | 2 | 3 | 5 | 1 |
| 1.027 | 4 | 1 | 1 | NT | 1 |
| 1.028 | 4 | 1 | 2 | NT | 1 |
| 1.029 | 2 | 1 | 1 | NT | 1 |
| 1.030 | 1 | 1 | 1 | NT | 1 |
| 1.031 | 1 | 1 | 1 | 1 | 1 |
| 1.032 | 3 | 1 | 4 | 3 | 1 |
| 1.033 | 2 | 1 | 1 | 1 | 1 |
| 1.034 | 5 | 2 | 5 | 5 | 3 |
| 1.035 | 4 | 3 | 5 | 5 | 3 |
| 1.036 | 1 | 1 | 1 | 1 | 1 |
| 1.037 | 1 | 1 | 1 | 1 | 1 |
| 1.039 | 5 | 3 | 5 | 5 | 1 |
| 1.040 | 5 | 4 | 5 | 5 | 2 |
| 1.041 | 1 | 1 | 1 | 1 | 1 |
| 1.042 | 5 | 4 | 5 | 5 | 1 |
| 1.043 | 5 | 1 | 4 | 3 | 1 |
| 1.044 | 2 | 1 | 3 | 3 | 1 |
| 1.046 | 1 | 2 | 1 | 1 | 1 |
| 1.047 | 5 | 5 | 5 | 5 | 3 |
| 1.048 | 4 | 2 | 5 | 4 | 1 |
| 1.049 | 5 | 3 | 5 | 5 | 1 |
| 1.050 | 1 | 1 | 1 | 1 | 1 |
| 1.051 | 5 | 3 | 5 | 4 | 2 |
| 1.052 | 5 | 3 | 5 | 4 | 1 |
| 1.054 | 4 | 1 | 5 | 4 | 1 |
| 1.055 | 4 | 1 | 4 | 4 | 1 |
| 1.056 | 4 | 1 | 4 | 3 | 1 |
| 1.057 | 2 | 2 | 4 | 2 | 1 |
| 1.058 | 2 | 2 | 3 | 3 | 1 |
| 1.059 | 2 | 2 | 2 | 2 | 1 |
| 1.060 | 4 | 4 | 4 | 4 | 2 |
| 1.061 | 3 | 1 | 5 | 4 | 1 |
| 1.062 | 4 | 1 | 4 | 2 | 1 |
| 1.063 | 3 | 2 | 2 | 2 | 1 |
| 1.064 | 1 | 1 | 1 | 1 | 1 |
| 1.065 | 2 | 2 | 4 | 3 | 1 |
| 1.066 | 3 | 2 | 5 | 4 | 1 |
| 1.067 | 3 | 2 | 4 | 3 | 1 |
| 1.068 | 5 | 2 | 5 | 4 | 2 |
| 1.069 | 3 | 2 | 5 | 3 | 1 |
| 1.070 | 5 | 3 | 4 | 4 | 1 |
| 1.071 | 5 | 3 | 5 | 5 | 2 |
| 1.072 | 4 | 1 | 4 | 2 | 1 |

N1.T = Not Tested.

1. A compound of Formula (I): 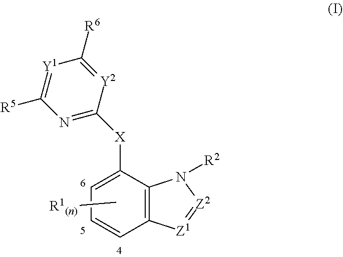
or an agronomically acceptable salt thereof,
wherein
X is selected from the group consisting of CH₂, O or S(O)ₚ;
Y¹ is N or CR³;
Y² is N or CR⁴;
with the proviso that Y¹ and Y² are not both N;
Z¹ is N or CR⁷;
Z² is N or CR⁸;
each R¹ is independently selected from the group consisting of halogen, —CN, nitro, C₁-C₄alkyl, C₂-C₄alkenyl, C₂-C₄alkynyl, C₁-C₄haloalkyl, C₁-C₄alkoxy-, C₁-C₄haloalkoxy-, —S(O)ₚC₁-C₄alkyl and —S(O)ₚC₁-C₄haloalkyl;
R² is selected from the group consisting of C₃-C₈ alkyl, C₃-C₈ alkenyl, C₃-C₈ alkynyl, C₃-C₈ haloalkyl, C₃-C₈ haloalkenyl, C₃-C₈ haloalkynyl, C₁-C₄alkoxy-C₁-C₃alkyl-, C₁-C₄haloalkoxy-C₁-C₃alkyl-, C₁-C₄alkoxy-C₁-C₃haloalkyl- and —(CH₂)ₘR⁹
R³ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁴ is selected from the group consisting of hydrogen, halogen, —CN, nitro, C₁-C₄alkyl, C₂-C₄alkenyl-, C₂-C₄alkynyl-, C₁-C₄haloalkyl-, C₁-C₄alkoxy-, C₁-C₄haloalkoxy- and —S(O)ₙC₁-C₄alkyl;
R⁵ is selected from the group consisting of hydrogen, halogen, C₁-C₃alkyl and C₁-C₃haloalkyl;
R⁶ is selected from the group consisting of hydrogen, halogen, C₁-C₃alkyl, C₁-C₃haloalkyl, C₁-C₃alkyl and C₁-C₃haloalkoxy;
R⁷ is selected from the group consisting of hydrogen, halogen, —CN, C₁-C₄alkyl, C₁-C₄haloalkyl- and C₁-C₄alkoxy-;
R⁸ is selected from the group consisting of hydrogen, halogen, —CN, C₁-C₄alkyl, C₁-C₄haloalkyl- and C₁-C₄alkoxy-;
R⁹ is selected from C₃-C₆ cycloalkyl, phenyl and a 5 or 6 membered heteroaryl which comprises from 1 to 4 heteroatoms each independently selected from the group consisting of oxygen, nitrogen and sulphur, and wherein said phenyl or heteroaryl groups are optionally substituted by one, two or three substituents independently selected from the group consisting of halogen, cyano, C₁-C₄alkyl, C₁-C₄haloalkyl, C₁-C₄alkoxy and C₁-C₄haloalkoxy;
m is 1, 2, 3 or 4
n=0, 1 or 2; and
p=0, 1 or 2.

2. A compound according to claim 1, wherein Y¹ is CR³ and Y² is N or Y¹ is CR³ and Y² is CR⁴ or Y¹ is N and Y² is CR⁴.

3. A compound according to claim 1, wherein Z¹ is CR⁷ and Z² is N or Z¹ is N and Z² is N or Z¹ is N and Z² is CR⁸.

4. A compound according to claim 1, wherein n=0.

5. A compound according to claim 1, wherein n=1 and R¹ is Cl or CN.

6. A compound according to claim 1, wherein R² is C₃-C₈ haloalkyl.

7. A compound according to claim 1, wherein R³ is halogen.

8. A compound according to claim 1, wherein R⁴ is selected from the group consisting of hydrogen, halogen and —CN.

9. A compound according to claim 1, wherein R⁵ is hydrogen.

10. A compound according to claim 1, wherein R⁶ is selected from the group consisting of hydrogen, C₁-C₄alkyl and C₁-C₃alkoxy.

11. A compound according to claim 1, wherein R⁷ is halogen.

12. A compound according to claim 1, wherein R⁸ is hydrogen.

13. A herbicidal composition comprising a compound according to claim 1 and an agriculturally acceptable formulation adjuvant.

14. A herbicidal composition according to claim 13, further comprising at least one additional pesticide.

15. A herbicidal composition according to claim 14, wherein the additional pesticide is a herbicide or herbicide safener.

16. A method of controlling weeds at a locus comprising application to the locus of a weed controlling amount of a composition according to claim 13.

17. Use of a compound of Formula (I) as defined in claim 1 as a herbicide.
